(19) United States
(12) Patent Application Publication
(10) Pub. No.: US 2026/0033972 A1
(43) Pub. Date: Feb. 5, 2026

(54) Orthopedic Sleeve

(71) Applicant: Aspen Medical Products, LLC, Irvine, CA (US)

(72) Inventors: Jane Price, Anaheim, CA (US); Joel Perez, Long Beach, CA (US); Frank Hernandez, Rancho Mission Viejo, CA (US)

(21) Appl. No.: 19/284,623

(22) Filed: Jul. 29, 2025

Related U.S. Application Data
(60) Provisional application No. 63/678,999, filed on Aug. 2, 2024.

(51) Int. Cl. A61F 5/01

(52) U.S. Cl. CPC A61F 5/0109

(57) ABSTRACT

An orthopedic sleeve comprising a sleeve body, and one or more tunnel segments formed within the sleeve body. Each of the one or more tunnel segments is configured to encapsulate a portion of a strap that is adapted to apply an unloading effect on a body part stabilized by the orthopedic sleeve.

### CROSS-REFERENCE TO RELATED APPLICATIONS

This application claims the benefit of priority on U.S. Provisional Application No. 63/678,999 filed Aug. 2, 2024 and U.S. Provisional Application No. 63/748,846 filed Jan. 23, 2025, the entire content of which are incorporated by reference herein.

### FIELD

Embodiment of the invention relate to orthopedic braces (orthoses), and in particular, knee braces.

### GENERAL BACKGROUND

Sleeve-based orthoses, while often lightweight and easy to apply, come with several limitations that can affect clinical effectiveness or impede continued usage. For example, Conventional sleeve-based orthoses are deployed with exterior straps and exterior hardgoods that are positioned over the exterior surface of the sleeve and outwardly exposed. The exterior straps and exterior hardgoods are intended to secure the orthosis to the leg and maintain alignment of the orthosis to avoid unwanted movement. However, the exterior exposure of the exterior straps and exterior hardgoods pose a number of issues, such as increased risk of snagging (e.g., catch on clothing or objects), tangling (e.g., due to washing, etc.), improper positioning of the straps relative to the leg, increased degradation caused by exposure to sunlight, moisture, and/or friction against coarse materials that may reduce the product lifecycle of the orthosis.

A sleeve-based orthosis that provides stability while eliminating the disadvantages associated with exposed exterior straps and exterior hardgoods are needed.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1A illustrates an exterior surface view of a first exemplary embodiment of a sleeve being part of an orthosis operating as a customized orthopedic brace.

FIG. 1B illustrates an interior surface view of the sleeve of FIG. 1A.

FIG. 2A illustrates a first side view of the sleeve integrated as part of an orthosis to form a sleeve-based orthosis placed into an operational state.

FIG. 2B illustrates a front perspective view of the sleeve integrated as part of the sleeve-based orthosis of FIG. 2A.

FIG. 2C illustrates a second side view of the sleeve integrated as part of the sleeve-based orthosis of FIG. 2A.

FIG. 2D illustrates a rear perspective view of the sleeve integrated as part of the sleeve-based orthosis of FIG. 2A.

FIG. 3A illustrates an exterior surface view of a second exemplary embodiment of a sleeve being part of an orthosis operating as a customized orthopedic brace.

FIG. 3B illustrates an interior surface view of the sleeve of FIG. 3A.

FIG. 3C illustrates an exterior surface view of a third exemplary embodiment of a sleeve being part of an orthosis operating as a customized orthopedic brace.

FIG. 4A illustrates a lateral perspective view of the sleeve integrated as part of an orthosis to form a sleeve-based orthosis placed into an operational state.

FIG. 4B illustrates a posterior perspective view of the sleeve integrated as part of the sleeve-based orthosis of FIG. 4A.

FIG. 5 illustrates a first embodiment of a sleeve-based orthosis, including orthotic components positioned along lateral or medial regions of the sleeve-based orthosis of FIGS. 2A-2D, including a hinge component and a plurality of cuffs including a first embodiment of a first cuff having a strap tensioning mechanism mounted thereon for concurrent tightening of the pair of straps.

FIG. 6 illustrates a first side (lateral) perspective view of the orthotic components of an embodiment of the sleeve-based orthosis of FIG. 5 when worn by a patient.

FIG. 7A illustrates a front perspective view of the sleeve-based orthosis of FIG. 5 when worn by a patient.

FIG. 7B illustrates a second side (medial or lateral) perspective view of the sleeve-based orthosis of FIG. 5 when worn by a patient.

FIG. 8 illustrates a second embodiment of a sleeve-based orthosis, including orthotic components positioned along lateral or medial regions of the sleeve-based orthosis of FIGS. 4A-4B, including a hinge component and a plurality of cuffs illustrated in FIG. 5.

FIG. 9A illustrates a front perspective view of the sleeve-based orthosis of FIG. 8 when worn by a patient.

FIG. 9B illustrates a side (medial or lateral) perspective view of the sleeve-based orthosis of FIG. 8 when worn by a patient.

FIG. 10A illustrates a third embodiment of a sleeve-based orthosis, including orthotic components positioned along lateral or medial regions of the sleeve-based orthosis, including a hinge component and a plurality of cuffs each including a strap tensioning mechanism mounted thereon for tightening of two straps with anterior direction of pull/tightening.

FIG. 10B illustrates a fourth embodiment of a sleeve-based orthosis, including orthotic components positioned along lateral or medial regions of the sleeve-based orthosis, including a hinge component and a plurality of cuffs each including a strap tensioning mechanism mounted thereon for tightening of two straps with posterior direction of pull/tightening.

FIG. 11A illustrates a lateral or medial perspective view of the sleeve-based orthosis of FIG. 10A or FIG. 10B placed into an operational state.

FIG. 11B illustrates a front perspective view of the sleeve-based orthosis of FIG. 11A.

FIG. 11C illustrates a posterior perspective view of the sleeve-based orthosis of FIG. 11A.

FIG. 12 illustrates a first embodiment of the first cuff having a strap tensioning mechanism mounted thereon and deployed within the sleeve-based orthoses of FIGS. 5 & 8.

FIG. 13A-13B illustrate a second embodiment of the first cuff having a strap tensioning mechanism mounted thereon and deployed within the sleeve-based orthoses of FIGS. 5 & 8.

FIGS. 14A-14B illustrate a third embodiment of the cuffs having strap tensioning mechanisms with different interconnect connectivity to support different strap controllability.

FIG. 15 illustrates mechanical components of the hinge component of FIGS. 5, 8, 10A-10B, including a hinge and a pair of members (e.g., strut members) coupled to the hinge.

FIG. 16A illustrates an overhead planar view of a first exemplary embodiment of the hinge component of FIG. 15 placed into a partially open (or unlocked) state.

FIG. 16B illustrates an overhead planar view of the hinge component of FIG. 15 placed into a closed (or locked) state.

FIG. 16C illustrates a laterally oriented perspective view of the hinge component of FIG. 16A.

FIG. 17A illustrates a perspective view of the hinge component of FIGS. 16A-16C oriented in a resting (non-flexed) state and the hinge placed in an open (or unlocked) state.

FIG. 17B illustrates a perspective view of the hinge component of FIGS. 16A-16C with the hinge placed in a closed (locked) state.

FIGS. 17C-17E illustrates perspective views of the hinge component of FIGS. 16A-16C oriented in a resting (non-flexed) state with the hinge component transitioning from a closed (locked) state to an open (or unlocked) state followed by removal of both an extension stop component, and a flexion stop component from the hinge component.

### DETAILED DESCRIPTION

Embodiments of the present disclosure generally relate to an orthosis, namely sleeve-based orthosis with one or more strap tensioning mechanisms. According to one embodiment of the disclosure, the sleeve-based orthosis features orthotic components, which are configured to control tensioning of straps that are fully encapsulated within an orthopedic sleeve (hereinafter, “sleeve”) of the sleeve-based orthosis that assist in providing an unloading effect to a body part (e.g., knee) supported by the orthosis. Herein, the sleeve-based orthosis will be described as an orthopedic knee brace; however, other orthopedic brace types may be configured with this described architecture and inventive aspects such as an arm or elbow brace.

For numerous embodiments of sleeve-based orthoses described below, the orthotic components may include, but are not limited or restricts to the following: (1) a dynamic unloading system featuring (1) a hinge component that includes (a) a hinge configured to allow for controlled movement of the knee joint, mimicking its natural motion to control extension and/or flexion, and/or (b) strut members coupled to the hinge that provide structural support to the orthosis; (2) one or more straps (e.g., any elongated material attached on both ends such as a band(s), cord(s), rope(s), etc.), which are adjustable and concentrate (e.g., crisscross, intersect, spiral, overlap, etc.) at a desired area of a patient's body (e.g., medial region of the leg towards the knee when the hinge is positioned on a lateral side of a patient's leg or a lateral region of the leg towards the knee when the hinge is positioned on a medial side of the patient's leg); and/or (3) cuffs installed at different region of the sleeve-based orthosis in which at least one cuff is adapted with a strap tensioning mechanism that allows for one or more interconnects (e.g., cable(s), wire(s), line(s), etc.) attached to one or more of the straps to be extended or retracted, which tightens or loosens the strap(s) that increases or reduces pressure and/or stress on the knee joint (referred to as “unload (ing) the knee”). Herein, an illustrative embodiment of a sleeve-based orthosis operating as a knee brace with a sleeve having pocket regions and tunnel segments for encapsulating the cuffs and strap(s) is illustrated below.

In general, one inventive aspect is directed to a sleeve for an orthosis, which features a sleeve body including one or more tunnel segments, whether knitted or sewn, to be part of the sleeve. Each tunnels segment is formed to encapsulate at least a portion of a strap. More specifically, when the sleeve is arranged and placed into a configuration to operate as an orthosis during use by a patient, the tunnel segment(s) may be oriented with at least one region of concentration such as a lateral or medial region offset from a patient's knee cap. For example, the sleeve may include a plurality of pocket regions including a first pocket region interconnected with a first set of tunnel segments and a second pocket region interconnected with a second set of tunnel segments. The first set of tunnel segments and the second set of tunnel segments are arranged to encapsulate different portions of the strap(s). At least a first pocket region maintains a tensioning mechanism to apply tension to the strap(s) housed by the first set of tunnel segments and the second set of tunnel segments that are arranged to unload a knee, namely re-distribute of forces away from or on the knee joint.

### I. Terminology

In the following description, certain terminology is used to describe aspects of the invention. For example, the term “formed” may be construed as knitted, sewn, welded, thermoformed, affixed (e.g., adhesive), or conducting another type of process to form open areas within material forming an object, such as sleeve portion of an orthosis. Herein, the term “knitted” with respect to an object (e.g., sleeve-based orthosis) is generally defined as any arrangement of material substantially comprising strands of material to produce that object. This “knitting” may constitute an attachment of the strands, which may constitute one or more thin, elongated lengths of one or more types of materials (e.g., natural, artificial such as thermoplastic material, etc.), together as a single layer of continuous material or as multiple (two or more) layers of material.

For instance, the knitting may be in accordance with a two-dimensional (2D) knitting process or a three-dimensional (3D) knitting process. The 2D knitting process allows for the creation of a low-profile orthoses where materials of different elasticity and different thermal thresholds are knitted in a single knitted layer of material. The 3D knitting process may be utilized to create a layered knitted orthosis, such as added padding and/or additional layers of the same or different types of materials to form tunnel segments and pocket regions.

Besides knitting, other attachment processes may include stitching (e.g., “V” shaped stitches) to attach two different pieces of material together, weaving (interlacing strands of material), crocheting (knot-like stitches), macramé (knot-like stitches in geometrical patterns), or another attachment scheme.

A “tunnel segment” is an area within the sleeve, normally elongated, for propagation of a portion of a strap within the sleeve to avoid significant exterior exposure of the strap. Likewise, a “pocket” is an area within the sleeve, normally with greater width than the tunnel segment, to retention of a cuff plate for a cuff, as described below.

As used herein, the term “semi-rigid” with respect to an object or portion of an object (e.g., a sleeve for an orthosis) means that the semi-rigid of the object will resist bending or deformation. This provides more rigidity than a flexible region, but the semi-rigid portion will be able to be bent and twisted at a substantial angle (e.g., greater than 45 degrees). The purpose of our semi-rigid region is to keep the orthosis (brace) from collapsing when worn. Similarly, the term “flexible” with respect to an object or a portion of an object means that the object or the object portion will not be permanently deformed or resistant to bending. For example, a knitted segment may be flexible in nature, even after a heating and cooling process. As used herein, the term “permanently deformed” means that deformation remains unless the deformation is actively repaired.

As used herein the term “patient” includes both humans and animals, independently of whether the patient is under the care of a medical or veterinary professional.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the orthosis may be configured to have a general tubular construction (e.g., an open top/bottom) or may be configured as a wrap that is placed into a general tubular construction when a first edge is wrapped around and attached to a surface of the orthosis as illustrated below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The character set “(s)” denotes one or more items. For example, the term “strap(s)” denotes one or more straps. The term “tunnel segment(s)” denotes one or more tunnel segments. The term “straps(s)” denotes one or more straps, and the like. The character set “/” between two reference numerals denotes “and/or” as defined below. For example, “100/110” denotes “100 and 110” or alternatively “100 or 110”.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

All methods or orthosis aspects described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various objects, features, aspects, and advantages of the inventive subject matter will become more apparent from the following detailed description of preferred embodiments, along with the accompanying drawing figures in which like numerals represent like components.

### II. General Architecture-Orthopedic Sleeve

The following discussion provides illustrative embodiments of different inventive subject matter. Although each embodiment may represent a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of properties such as sizing, degrees of motion, and so forth, may be described in connection with certain embodied inventive aspect, but these quantities may be modified while still retaining the spirit and scope of the disclosed invention. Accordingly, in some embodiments, the numerical parameters set forth in the written description are illustrative approximations that can vary depending upon the desired properties sought to be obtained by a particular embodiment.

Groupings of inventive embodiments disclosed herein are not to be construed as limitations. Each inventive embodiment, as well as each illustrative component or feature within an inventive embodiment, can be referred to and may be claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

### A. First Orthopedic Sleeve Embodiment

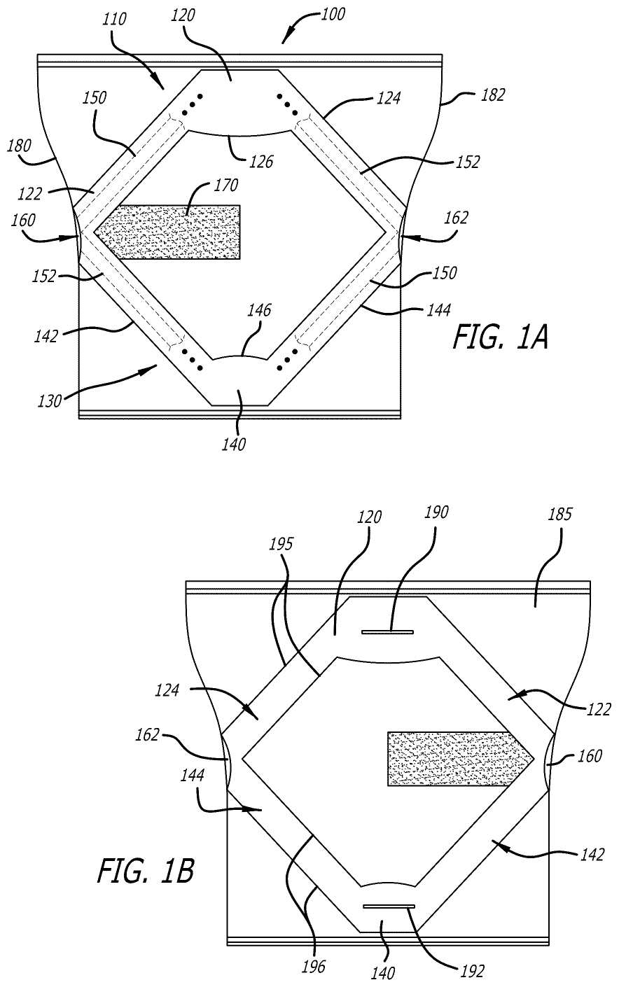

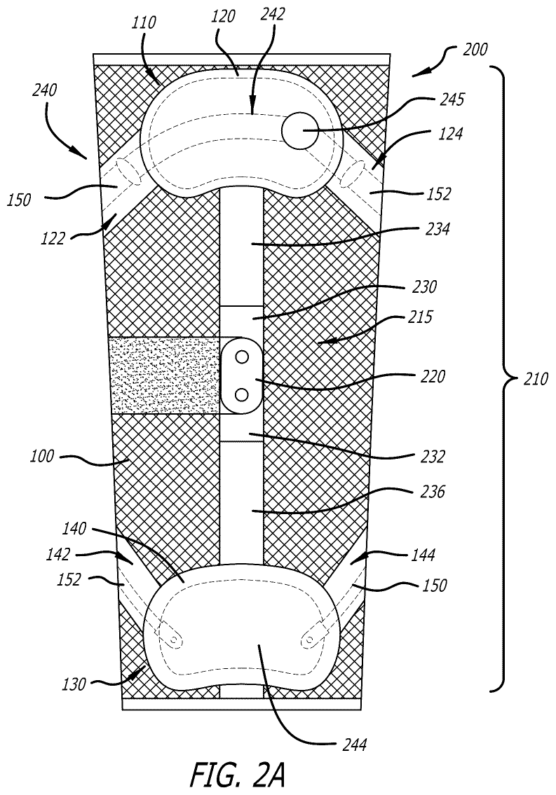

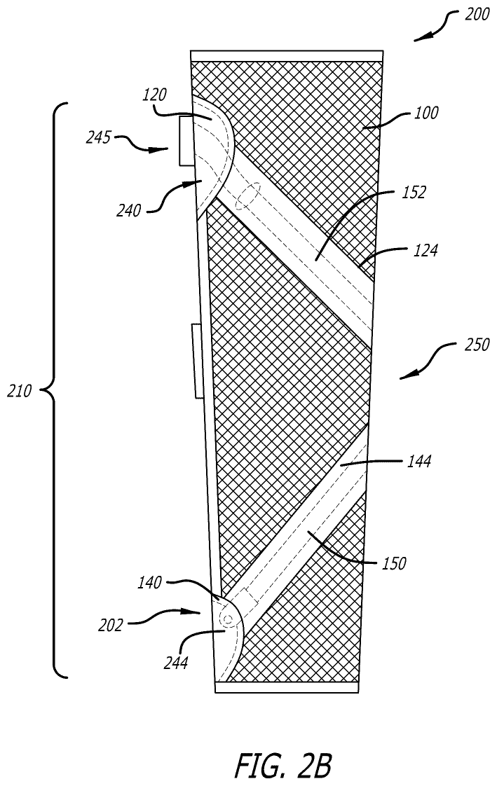

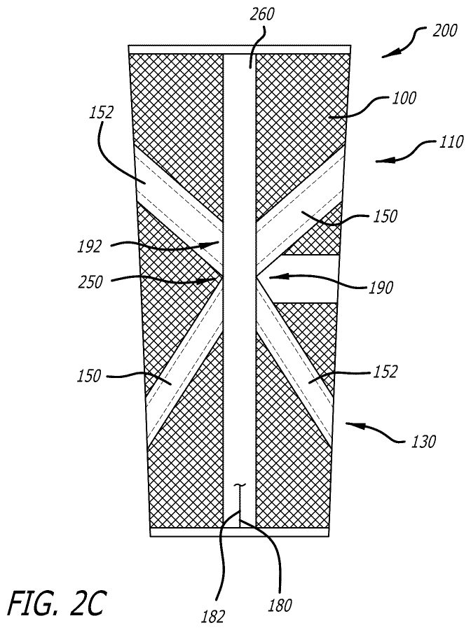

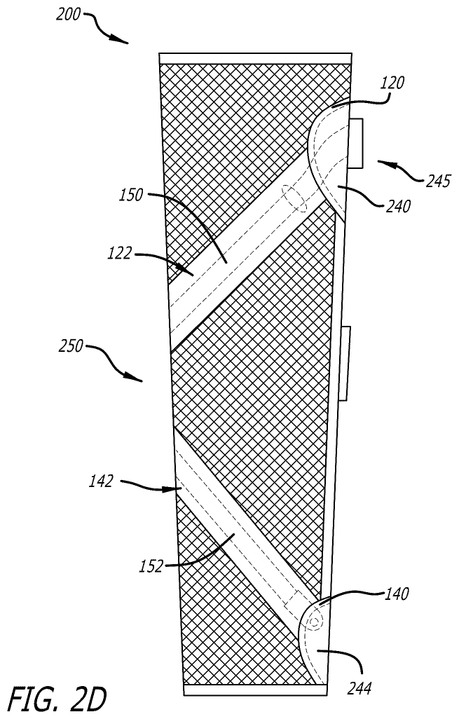

Referring to FIG. 1A, an exterior surface view of a first exemplary embodiment of a sleeve 100 being part of an orthosis operating as an orthopedic brace is shown. Herein, the sleeve 100 features a sleeve body including a first pathway 110 and a second pathway 130. The first pathway 110 includes a first pocket region 120 communicatively coupled to a first tunnel segment 122 and a second tunnel segment 124. The second pathway 130 includes a second pocket region 140 communicatively coupled a third tunnel segment 142 and a fourth tunnel segment 144. The first and second pathways 110 and 130 may be created as areas formed by spatial separation within the sleeve 100, where the spatial separation may be achieved by a prescribed knitting technique (e.g., inlay of material to create hollow regions, creation of looser or tighten knit areas, etc.), by different stitching types, or by sewing overlaying layers of materials together to form the pathways 110 and 130. A first slit 126 may be formed along an edge or on top of the first pocket region 120 (for receipt of the first strut member, see FIG. 2A), while a second slit 146 may be formed along an edge or on top of the second pocket region 140 (for receipt of the second strut member, also see FIG. 2A).

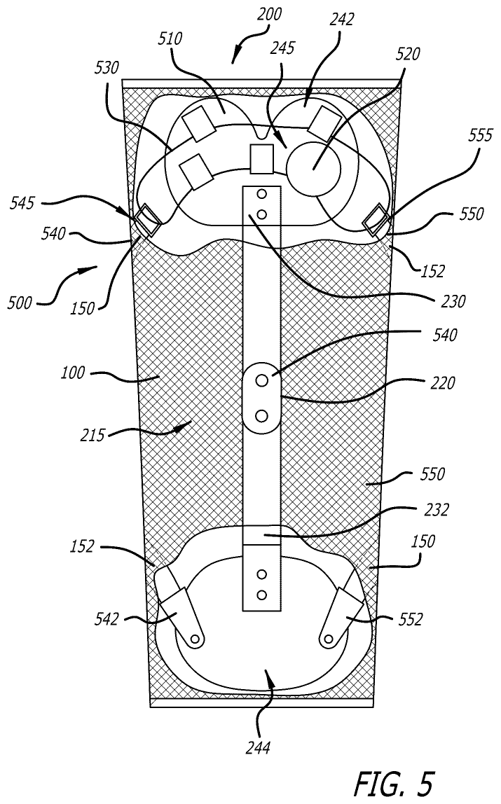

According to one embodiment of the disclosure, the first tunnel segment 122 and the second tunnel segment 124 extend from the first pocket region 120, where the first tunnel segment 122 is sized to encapsulate an upper portion of a first strap 150 and the second tunnel segment 124 is sized to encapsulate an upper portion of a second strap 152. One or both of these straps 150 and 152 are coupled to interconnects that are adjusted by one or more strap tensioning mechanisms to control the degree of extension (or retraction) and the amount of tension to unload a body part (e.g., knee) stabilized by the sleeve 100 as shown in FIGS. 5 & 10A-10B. Stated differently, the first strap 150 and the second strap 152 may be tightened or loosened by a strap tensioning mechanism (not shown) having one or more interconnects coupled to the strap(s) 150 and/or 152 situated within the pathways 110 and 130.

Similar to the first pathway 110, the second pathway 130 features the third tunnel segment 142 and the fourth tunnel segment 144, which are communicatively coupled to and extend from the second pocket region 140. The third tunnel segment 142 is sized to encapsulate a lower portion of the second strap 152 while the fourth tunnel segment 144 is sized to encapsulate a lower portion of the first strap 150.

Additionally, as shown in FIG. 1A, a first opening 160 is situation at a point of intersection between the first tunnel segment 122 and the third tunnel segment 142. Similarly, a second opening 162 is situation at a point of intersection between the second tunnel segment 124 and the fourth tunnel segment 144. The first opening 160 and the second opening 162 provide access to the first strap 150 and the second strap 152 for assembly, removal for replacement or repair.

The sleeve 100 may have different physical characteristics located at different regions of the sleeve 100. For instance, an offset region 170 may be considered with a different stitching pattern, different stitch thicknesses, and/or different material types (e.g., flexible material with the remainder of the material forming the sleeve 100 being semi-rigid material). When the sleeve 100 operates as part of a sleeve-based orthosis placed into an operational state as shown in FIGS. 2A-2B, the offset region 170 is positioned behind the knee. Similarly, thermoplastic material may be positioned proximate to the edges 180 and 182 of the sleeve 100. The thermoplastic material coupled to configured with a thermal threshold lesser than the material forming the remainder of the sleeve 100 so that, after applying heat to partially melt the thermoplastic material, the region of the sleeve with the thermoplastic material becomes more rigid to support and retain the cylindrical structure of the sleeve 100 when placed into operational state.

Referring to FIG. 1B, an interior surface view of the sleeve 100 of FIG. 1A is shown. Herein, the interior surface 185 provides access to the pocket regions 120 and 140 via a first pocket slit 190 and the second pocket slit 192, respectively. Furthermore, the pathways 110 and 130 are formed within the sleeve 100, where edges 195 and 196 of the pathways 110 and 130 are arranged to provide the first pocket region 120, the second pocket region 140, and the plurality of tunnel segments 122-124 and 142-144.

Referring now to FIG. 2A, an exemplary first side view of the sleeve 100, which is integrated as part of an orthosis and configured to form a sleeve-based orthosis 200 when placed into an operational state, is shown. Herein, the edges 180 and 182 of the sleeve 100, as shown in FIG. 1A, are re-positioned towards each other so that the sleeve-based orthosis 200 possesses a substantially cylindrical form as shown. As a result, the first pocket region 120 of the first pathway 110, which are not shown in detail to allow for clarity of the strap interconnectivity with a pair of cuffs 240, is intended to be positioned above the knee (e.g., placed over the femur) and the second pocket region 140 of the second pathway 130 is intended to be positioned below the knee (e.g., resting over the tibia).

The sleeve-based orthosis 200 features a dynamic unloading system 210, which includes a hinge component 215 and the pair of cuffs 240, which is coupled to the hinge component 215 and respectively positioned within the first pocket region 120 and the second pocket region 140. Herein, the hinge component 215 is interposed between the first pocket region 120 and the second pocket region 140 of the sleeve 100. According to one embodiment of the disclosure, the hinge component 215 includes (i) a hinge 220 configured to allow for controlled movement of the knee joint, mimicking its natural motion to control extension and/or flexion, and/or (ii) the pair of strut members 230/232 coupled to the hinge 220. The strut members 230/232 may include welded tunnels 234/236, such as UBL fabrics that are welded onto the knit to create tunnels for the strut members 230/232 for example. For this structure, the dynamic unloading system 210 is configured to provide structural support to the sleeve-based orthosis 200.

As further shown in FIG. 2A, the pair of cuffs 240 includes a first cuff 242 and a second cuff 244. The first cuff 242 features a strap tensioning mechanism 245, which is adapted to concurrently (and in fact simultaneously) adjust the tension of the first strap 150 positioned within the first tunnel segment 122 and the fourth tunnel segment 144 and the second strap 152 positioned within the second tunnel segment 124 and the third tunnel segment 142. As a result, these adjustable straps 150/152 are positioned to concentrate at a region of the sleeve 100 opposite to the hinge 220. For example, where the hinge 220 is located on the first side of the sleeve-based orthosis 200, the adjustable straps 150/152 may be configured to intersect at the second side of the sleeve-based orthosis 200. As shown in FIG. 2C, for this illustrative embodiment, the adjustable straps 150/152 crisscross at a mid-medial or lateral region 250 of the sleeve-based orthosis 200 to provide an unload effect to the knee of the patient when the sleeve-based orthosis 200 is worn.

Referring to FIG. 2B, a front perspective view of the sleeve 100 integrated as part of the sleeve-based orthosis 200 of FIG. 2A is shown. For this embodiment, as shown, the dynamic unloading system 210 is positioned on a lateral side 202 of the sleeve-based orthosis 200 when placed on a right leg of the patient. However, given its pseudo-universal design, the dynamic unloading system 210 would be positioned on a medial side of the sleeve-based orthosis 200 when placed on a left leg of the patient, where this perspective view would be rearward perspective view of the sleeve-based orthosis 200.

As shown, for this embodiment, the second tunnel segment 124 extends diagonally downward from the first pocket region 120 toward the mid-medial region 250 of the sleeve-based orthosis 200. Also, the fourth tunnel segment 144 extends diagonally upward from the second pocket region 140 toward the mid-medial region 250 of the sleeve-based orthosis 200. Complementary in orientation, as shown in FIG. 2D, the first tunnel segment 122 extends diagonally downward from the first pocket region 120 toward the mid-medial region 250 while the third tunnel segment 142 extends diagonally upward from the second pocket region 140 toward the mid-medial region 250 of the sleeve-based orthosis 200. As illustrated in FIGS. 2B & 2D, this configuration enables the first strap 150 to be adjustably coupled to the strap tensioning mechanism 245 mounted on the first cuff 240 maintained in the first pocket region 120, fixedly coupled to the second cuff 244 maintained within the second pocket region 140, and propagate within the first tunnel segment 122 and the fourth tunnel segment 144.

Referring to FIG. 2C, a second side view of the sleeve 100 integrated as part of the sleeve-based orthosis 200 of FIG. 2A is shown. Herein, the edges 180 and 182 of the sleeve 100 are re-positioned towards to other so that the sleeve-based orthosis 200 possesses a substantially cylindrical form as shown. As a result, the openings 160/162 formed on the pathways 110 and 130 are aligned to provide an intersection point between the first strap 150 and the second strap 152, where the adjustable straps 150/152 crisscross at the mid-medial region 250. Although not shown, fasteners may be attached to the exterior surface of the sleeve 100 to retain in its substantially cylindrical form. Additionally, or in the alternative, an optional band of thermoplastic material 260 may be positioned longitudinally along the second side (e.g., medial side) to provide additional support after the thermoplastic material is heated above its thermal melting threshold and subsequent cooling to transform into a semi-rigid form to provide additional stability against medial movement of the knee during an activity.

### B. Second Orthopedic Sleeve Embodiment

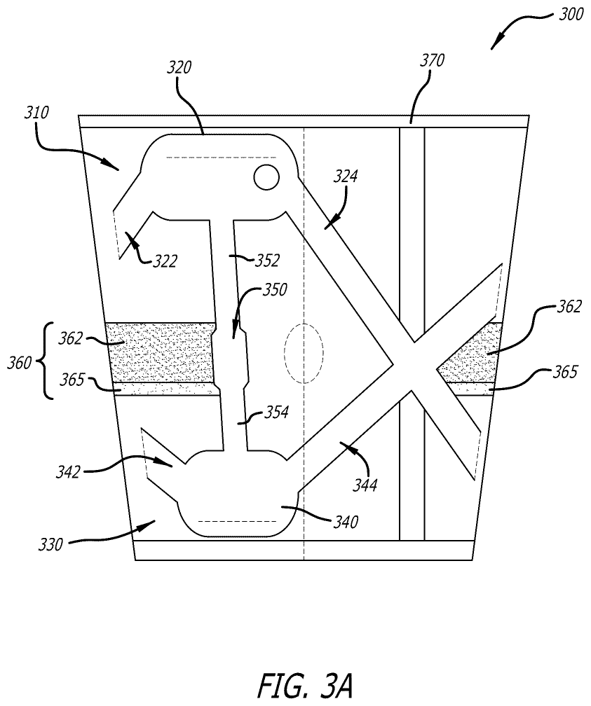

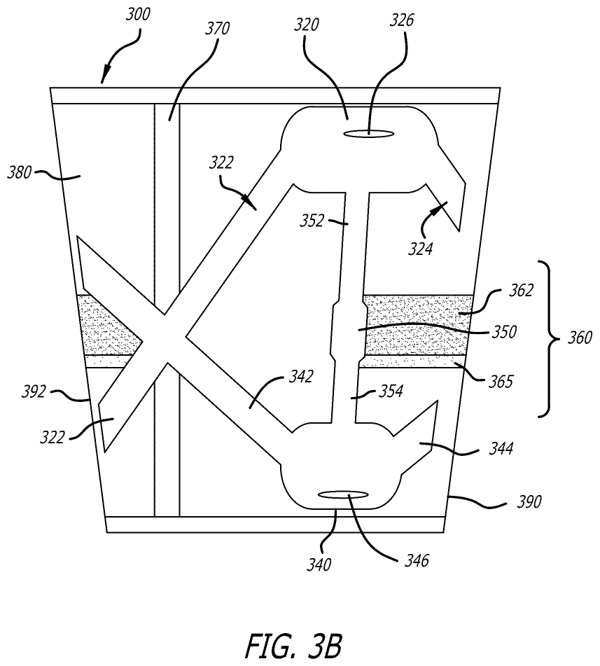

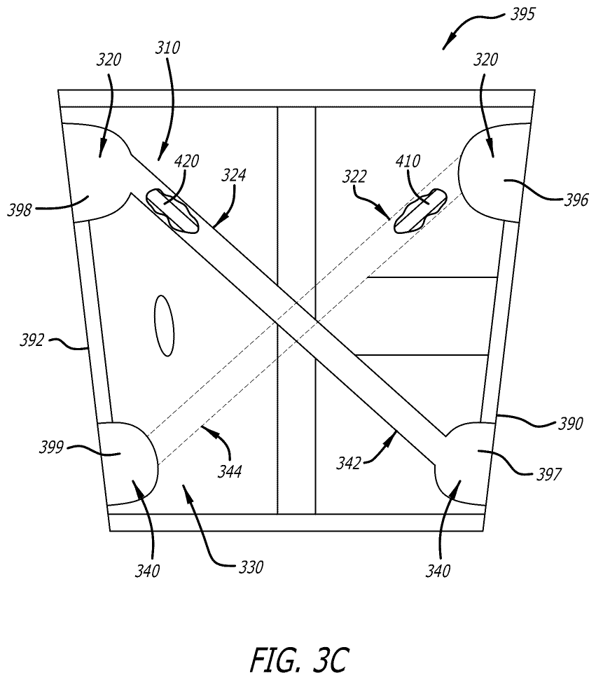

Referring to FIG. 3A, an exterior surface view of a second exemplary embodiment of a sleeve 300 being part of an orthosis operating as an orthopedic brace is shown. Herein, the sleeve 300 features a sleeve body including a first pathway 310 and a second pathway 330. The first pathway 310 includes a first pocket region 320 communicatively coupled to a first tunnel segment 322 and a second tunnel segment 324. The second pathway 330 includes a second pocket region 340 communicatively coupled a third tunnel segment 342 and a fourth tunnel segment 344.

Spatially separated from each other, the first and second pathways 310 and 330 are open areas formed within sleeve 300, where the formation of the first and second pathways 310 and 330 are achieved through prescribed knitting techniques to create such open areas, different stitching types, or sewn overlaying layers of materials to form the pathways 310 and 330.

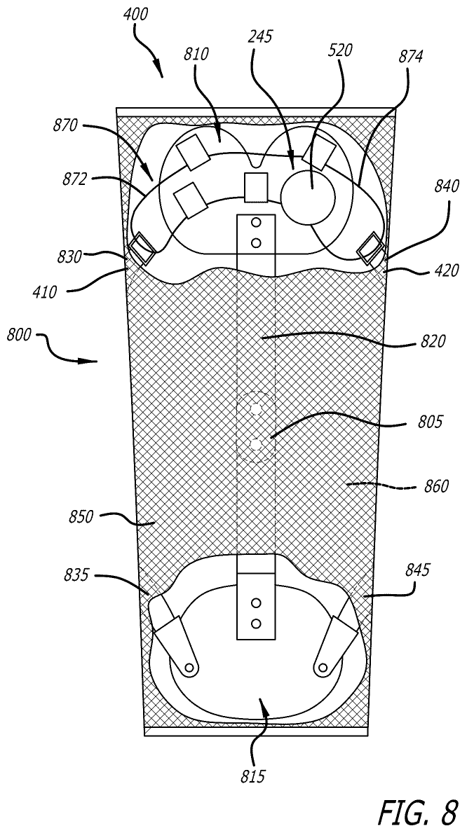

According to this embodiment of the disclosure, as shown in FIG. 3A, the first tunnel segment 322 and the second tunnel segment 324 of the sleeve 300 extend from the first pocket region 320, where the first tunnel segment 322 is sized to encapsulate an upper portion of a first strap (not shown) and the second tunnel segment 324 is sized to encapsulate an upper portion of a second strap (not shown). One or both of these straps are coupled to one or more strap tensioning mechanisms to control the degree of extension (or retraction) and the amount of tension to distribute load or unload a body part stabilized by the sleeve 300 (e.g., knee) as shown in FIGS. 8 & 9A-9B.

Additionally, as shown in FIG. 3A, the sleeve 300 further includes a third pocket region 350 for the hinge 220 of FIG. 2A along with tunnel segments 352 and 354 sized for encapsulating the strut members 230/232 of FIG. 2A. The sleeve 300 may have an offset area 360 with different characteristics 362 (e.g., different stitching patterns or thicknesses) and/or different material types 365 (e.g., flexible material, thermoplastic material, etc.). A longitudinal region 370 of thermoplastic material may be configured on a side (lateral or medial) region of the sleeve, when formed as part of the orthosis, to provide additional stability and resistance against side (lateral or medial) directed forces once the material has been heated to toward melting temperature and cooled to harden.

Referring now to FIG. 3B, an interior surface view of the sleeve 300 of FIG. 3A. Herein, an inner surface 380 of the sleeve 300 features the first pocket region 320 with a first slit 326 sized to receive the first cuff 242, the second pocket region 340 with a second slit 346 sized to receive the second cuff 244, the third (hinge) pocket region 350, tunnel segments 322, 324, 342, 344, 352 & 354, and longitudinal region 370 for the thermoplastic material, and the like. When the sleeve 300 is configured as the sleeve-based knee orthosis, the offset area 360 would be positioned behind the knee cap, where the offset area 360 features materials of the different characteristics 362 (e.g., different stitching patterns or thicknesses) and/or different material types 365 (e.g., flexible material, thermoplastic material, etc.) than the remainder of the sleeve 300.

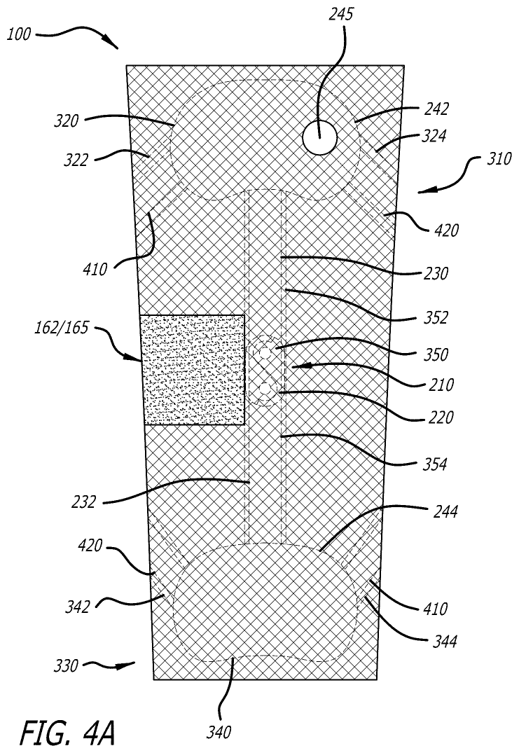

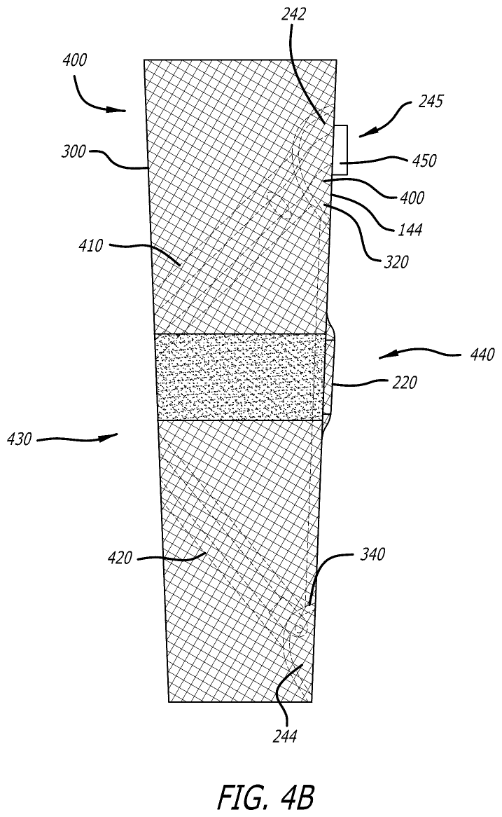

To be placed into operational state to operate as part of a sleeve-based orthosis 400 of FIGS. 4A-4B, the edges 390 and 392 are folded toward each other so that the sleeve 300 would have a cylindrical form. In the operational state, the first tunnel segment 322 is downwardly oriented and becomes aligned with the fourth tunnel segment 344, which is upwardly oriented. Also, the second tunnel segment 324 is downwardly oriented and becomes aligned with the third tunnel segment 342 that is upwardly oriented.

Referring now to FIG. 3C, an exterior surface view of a third exemplary embodiment of a sleeve 395 being part of an orthosis operating as an orthopedic brace is shown. Herein, the sleeve 395 features the sleeve body including the first pathway 310 and the second pathway 330 similar to the sleeve 300 of FIG. 3A, except for the pocket configuration and/or strap configuration. More specifically, the first pocket region 320 and the second pocket region 340 are segmented in which a first portion 396 of the first pocket region 320 and a first portion 397 of the second pocket region 340 are configured to border by the first edge 390. Additionally, a second portion 398 of the first pocket region 320 and a second portion 399 of the second pocket region 340 are configured to border by the second edge 392.

Upon folding to attach the edges 390/392 to form the sleeve-based orthosis, the tunnel segments 322/324/342/344 form open tunneling paths between the pocket regions 320/340. It is contemplated that, in lieu of two tunneling paths, namely tunnel segments 322/344 and 324/342 that are adapted to encapsulate straps 410 and 420, it is contemplated that only one tunneling path may be utilized to encapsulate a corresponding strap (e.g., strap 420 or 410). As shown, tunnel segments 324/342 may be utilized to encapsulate strap 420 to which tension is applied to effectuate an unloading effect on the patient's knee, where optional tunnel segments 322/344 (shown by dashed lines), when formed as part of the sleeve 395, may be utilized to encapsulate strap 410. Hence, the strap tensioning mechanism may be configured to control the application of tension to the second strap 420 encapsulated in tunnel segment(s) 324/342 and/or the application of tension to the first strap 410 encapsulated in tunnel segment(s) 322/344.

Referring to FIG. 4A, an exemplary first side view of the sleeve 300, which is integrated as part of an orthosis to form a sleeve-based orthosis 400 and placed into an operational state, is shown. Herein, the edges 390 and 392 of the sleeve 300 are folded towards to other so that the sleeve-based orthosis 400 possesses a substantially cylindrical form as shown. As a result, the first pocket region 320 of the first pathway 310 is intended to be positioned above the knee (e.g., placed over the femur) and the second pocket region 340 is intended to be positioned below the knee (e.g., resting over the tibia).

The sleeve-based orthosis 400 features the dynamic unloading system 210 of FIG. 2A. which is interposed between the first pocket region 320 and the second pocket region 340 of the sleeve 300 and encapsulated within the third pocket region 350 and tunnel segments 352 and 354 formed within the sleeve 300. The dynamic unloading system 210 includes the hinge 220 and strut members 230/232.

The pair of cuffs 240, namely the first cuff 242 and the second cuff 244 of FIG. 2A, are positioned within the first pocket region 320 and the second pocket region 340, respectively. As shown, the first cuff 242 features the strap tensioning mechanism 245, which is adapted to concurrently adjust the tension of a first strap 410 positioned within the first tunnel segment 322 and the fourth tunnel segment 344 and the second strap 420 positioned within the second tunnel segment 324 and the third tunnel segment 342. Similar to the layout of the sleeve 100 of FIGS. 1A-1B, these adjustable straps 410/420 are positioned to concentrate at a region of the sleeve 300 opposite to the hinge 220. For example, where the hinge 220 is located on the first side of the sleeve-based orthosis 400, the adjustable straps 410/420 intersect at the second side of the sleeve-based orthosis 200. As shown in FIG. 4B, the adjustable straps 410/420 crisscross at a mid-medial region 430 of the sleeve-based orthosis 400, which is opposite to the mid-lateral region 440 at which the hinge 220 is positioned, to load/unload forces on the knee of the patient.

Referring now to FIG. 4B, a posterior perspective view of the sleeve-based orthosis 400 of FIG. 3A is shown. The strap tensioning mechanism 245 is located on the first cuff 242, which is at least partially encapsulated within the first pocket region 320. Based on rotation of a control knob 450 extending from a curved surface of the first cuff 242, a length of an interconnect (cable) that is attached to upper ends of the straps 410/420 may be decreased or increased, which causes tensioning or de-tensioning of the straps 410/420. As shown in FIG. 4A and described in greater detail in FIG. 8, given that the first strap 410 is integrated in the first tunnel segment 322 and coupled at one end to the interconnect, the retraction of the interconnect may cause increased tension of the straps 410/420 and result in increased pressure on a lateral or medial region of the patient's leg upon which the straps are concentrated. In contrast, the extension of the interconnect causes a reduction in tension of the straps realized on the lateral or medial regions of the patient's leg.

### III. General Architecture-Sleeve-Based Orthoses

### A. First Sleeve-Based Orthosis Embodiment

Referring now to FIG. 5, a first embodiment of the sleeve-based orthosis 200 of FIGS. 2A-2D is shown, which includes orthotic components 500 positioned along lateral or medial regions of the sleeve-based orthosis 200. The orthotic components 500 include, but are not limited or restricted to the hinge component 215 and the plurality of cuffs 240 including the first cuff 242 (e.g., a first embodiment of a first cuff) having the strap tensioning mechanism 245 mounted thereon for concurrent tightening of the first strap 150 and the second strap 152. While the cuffs 242 and 244 are inserted into pocket regions 120 and 140 and the straps 150 and 152 are inserted into tunnels 122/144 and 124/142 of the sleeve 100, a cuff-away view is provided to illustrate details of the cuff/strut/strap connectivity.

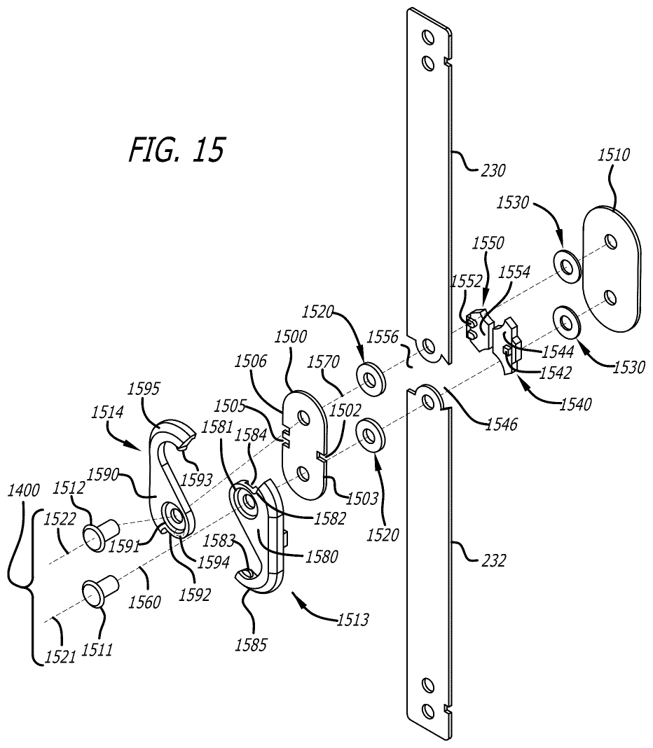

As shown in FIG. 5, the hinge component 215 includes (i) the hinge 220 configured to allow for controlled movement of the knee joint, mimicking its natural motion to control extension and/or flexion, and/or (ii) the pair of strut members 230/232 coupled to the hinge 220. The first strut member 230 is fixedly coupled to the first cuff 242 and pivotally coupled to one or more components of the hinge 220 as described below and illustrated in FIG. 15. The second strut member 232 is fixedly coupled to the second cuff 244 and pivotally coupled to component(s) of the hinge 220.

Herein, the strap tensioning mechanism 245 is mounted on a top surface 510 of the first cuff 242, where the strap tensioning mechanism 245 includes a control knob 520 that is used to adjust the degree of extension (or retraction) of the overall length of the entire strap body (interconnect and strap) and the amount of tension applied to the first strap 150 and the second strap 152. More specifically, as shown, the interconnect 530 is arranged in a loop configuration, where the length of the interconnect 530 is adjusted based on the rotational setting of the control knob 520. As shown, a first end 540 of the first strap 150 is coupled to a first attachment member 545 (e.g., D-ring) while a second end 552 of the first strap 150 is fixedly secured to the second cuff 244. Similarly, a first end 550 of the second strap 152 is coupled to a second attachment member 555 (e.g., D-ring) while a second end 542 of the second strap 152 is fixedly secured to the second cuff 244.

Based on this architecture, the shortening of the looped interconnect 530 causes extension of the first strap 150 and the second strap 152 and increased tension is applied by the straps 150/152 to increase the unloading effect on the knee (e.g., reduce pressure on a specific part of the knee joint patient's body and/or shift the load away from an injured or weaker part of the knee). Conversely, lengthening of the looped interconnect 530 causes retraction of the first strap 150 and the second strap 152 towards its rest state thereby lessening the unloading effect.

It is contemplated that the placement of the first cuff 242 and the second cuff 244 can be reversed, where the strap tensioning mechanism 245 would be positioned below the patient's knee. Also, the hinge component 215 is described in detail in FIG. 15, albeit it is contemplated that other hinge types may be utilized such as a polycentric hinge.

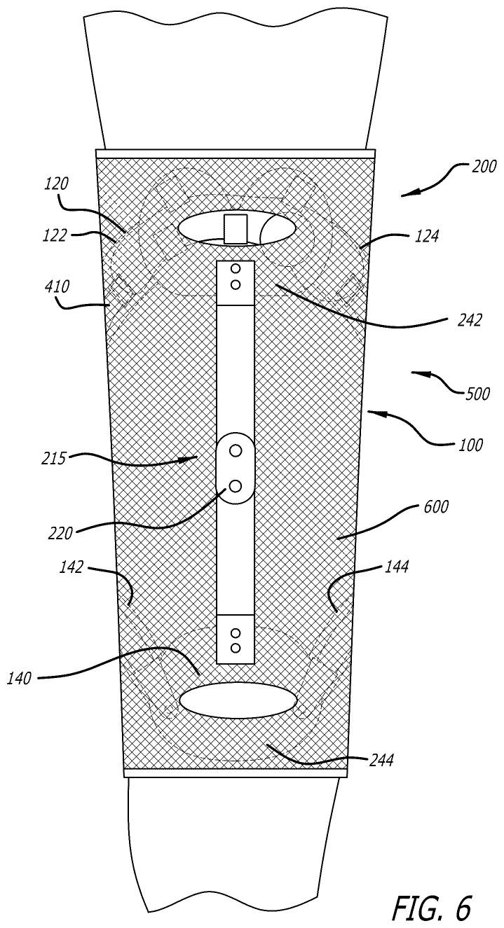

Referring to FIG. 6, a first side (lateral) view of the orthotic components 500 of an embodiment of the sleeve-based orthosis 200 of FIG. 5, when worn by a patient, is shown. Herein, the first cuff 242 is inserted into the first pocket region 120 to prevent the interconnect 530 from being accidentally snagged and potentially damaged during use. The second cuff 244 is inserted into the second pocket region 140. As an optional feature, the hinge component 215 is positioned over an exterior surface 600 of the sleeve 100 to allow for easier adjustment of the hinge 220 to control the selected degrees of extension and/or flexion.

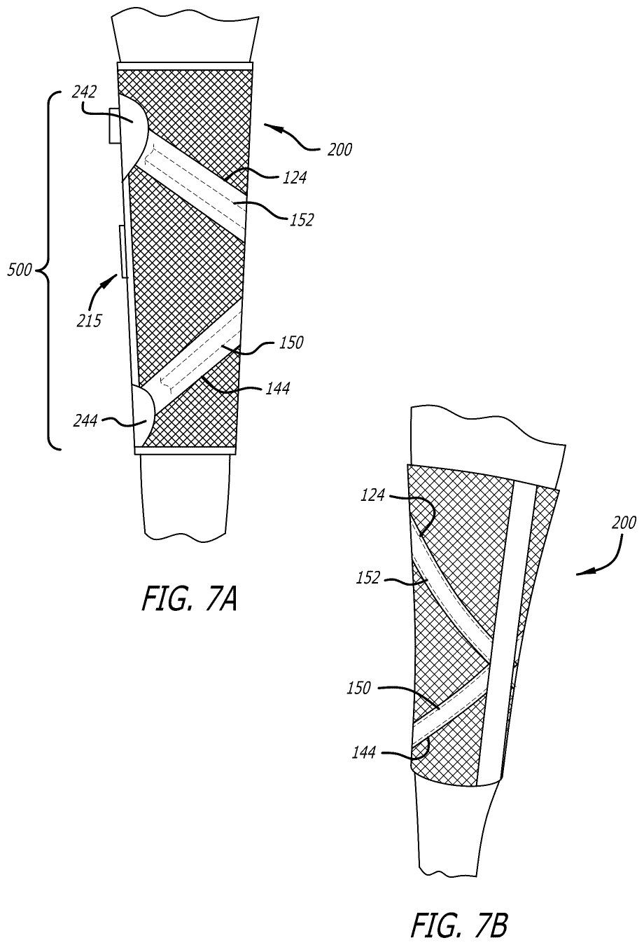

Referring now to FIG. 7A, a front perspective view of the sleeve-based orthosis 200 of FIG. 5, when worn by a patient, is shown. Herein, the orthotic components 500, inclusive the hinge component 215 the first and second cuffs 242 and 244, is positioned on a first side (e.g., lateral region) of the sleeve-based orthosis 200 as the sleeve-based orthosis 200 is shown to be worn on the patient's right leg while the orthotic components 500 would be positioned on the medial region when worn on the patient's left leg. Additionally, illustrated in a cut-away view, a substantial portion (or the entirety) of the first and second straps 150 and 152 (e.g., 90% of the strap lengths) are encapsulated within prescribed tunnel segments 122/144 and 124/142 formed within the sleeve 100, with an intersection point positioned on a second side of the sleeve-based orthosis 200 opposite from the first side with the orthotic components 500. For this illustrative embodiment, the hinge 220 of the hinge component 215 is positioned on a lateral region of the patient's leg while the intersecting straps 150/152 are located on a medial region of the patient's leg.

For example, as shown in FIG. 7B, a second side perspective view of the sleeve-based orthosis 200 of FIG. 5 is shown. Herein, a lateral perspective view is shown as the first strap 150 and the second strap 152 are arranged to provide a crisscross orientation for the first strap 150 and the second strap 152. Alternatively, when the sleeve worn on an opposite leg, the cross-crossing of the straps 150 and 152 would be positioned on a lateral side of the leg. While the crisscross orientation for the first strap 150 and the second strap 152 provides an effective orientation to apply an unloading effect to the patient's knee, it is contemplated that one or more of the tunnel segments 122 124, 142, and/or 144 of FIG. 5 may be arranged to provide any orientation (e.g., spiral, etc.) so long as the orientation is suitable to provide sufficient unloading effect to the patient's knee when the sleeve-based orthosis 200 is worn.

### B. Second Sleeve-Based Orthosis Embodiment

Referring now to FIG. 8, a second embodiment of a sleeve-based orthosis, represented in detail as the sleeve-based orthosis 400 of FIG. 4A, is shown. Herein, the sleeve-based orthosis 400 includes orthotic components 800 positioned along lateral or medial regions of the sleeve-based orthosis 400. The orthotic components 800 include, but are not limited or restricted to (i) a hinge 805, (ii) first and second cuffs 810 and 815, (iii) a first (geared) strut member 820 coupled to hinge 805 and the first cuff 810, and/or (iv) a second (geared) strut member 825 coupled to the hinge 805 and the second cuff 815. Shown in a cut-away view of the pocket regions 320/340 of the sleeve 300 to better illustrate the strap connectivity with the cuffs 810/815, for this embodiment, all of the orthotic components 800 inclusive of the straps are encapsulated within areas of the sleeve 300.

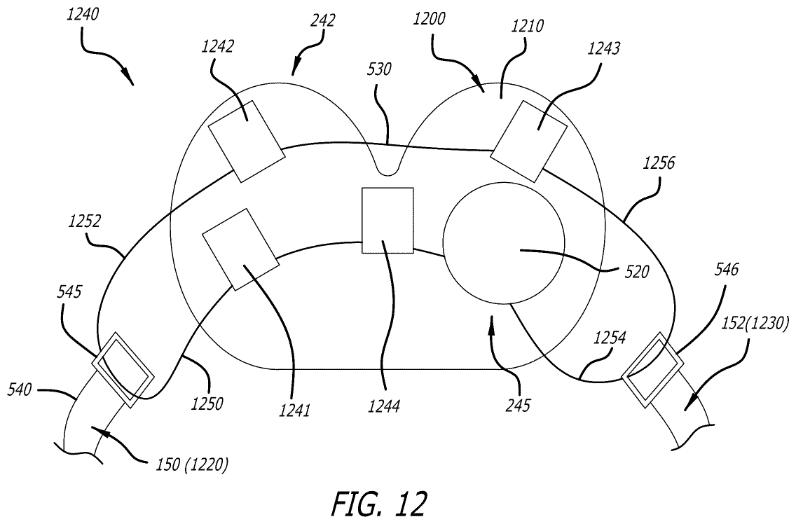

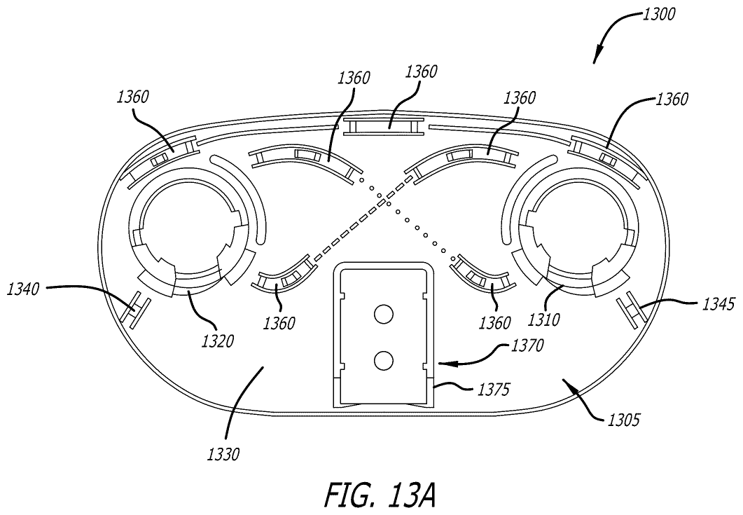

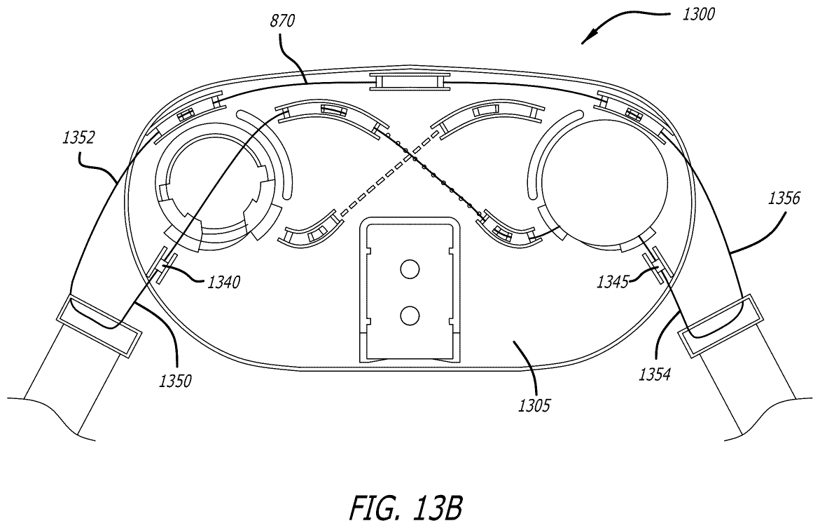

Additionally, the first strap 410 features a first end 830 coupled to the strap tensioning mechanism 245 mounted on the first cuff 810 and a second end 835 coupled to the second cuff 815. The second strap 420 features a first end 840 also coupled to the strap tensioning mechanism 245 and a second end 845 coupled to the second cuff 815. Herein, the hinge 805 may be constructed as the hinge 220 of FIG. 2A as shown, or alternatively, the hinge 805 may be implemented with another hinge design such as a polycentric hinge. Also, the first and second cuffs 810 and 815 may be constructed as cuff 242 and 244 of FIG. 12 as shown (see FIGS. 2A & 4A), or alternatively, the first cuff 810 may be implemented with another cuff design such as a cuff with prescribed interconnect routing within the cuff plate as shown in FIGS. 13A-13B.

More specifically, as illustrated in FIG. 8, the first and second straps 410 and 420 extend across a second side 860 (e.g., medial or lateral side) of the sleeve-based orthosis 400, where the second side 860 of the sleeve-based orthosis 400 opposite a first side 850 (e.g., lateral or medial side) featuring the orthotic components 800. The first strap 410 is coupled to the first cuff 810 via an interconnect 870, which is shortened and lengthened based on actuation of the control knob 520 of the strap tensioning mechanism 245. The actuation may be conducted by rotation of the control knob 520, although other actuation processes may be conducted for de-tensioning of the strap such as pulling on the control knob 520 which releases tension.

In particular, conducting a first operation on the strap tensioning mechanism 245 (e.g., actuation of the control knob 520 of the strap tensioning mechanism 245 such as rotation in a first angular (clockwise, CW) direction) causes winding of the interconnect 870, which reduces the length of a first segment 872 of the interconnect 870 coupled to the first end 830 of the first strap 410 and pulls the first strap 410 towards the first cuff 810. Concurrently, actuation of the strap tensioning mechanism 245 (e.g., rotation in the first angular direction causing winding of the interconnect 870) further causes shortening of a second segment 874 of the interconnect 870 coupled to the first end 840 of the second strap 420 and pulls the second strap 420 towards the first cuff 810. This effectively increases tensioning of the straps 410 and 420 that may increase an unloading effect on the knee of the patient.

Similarly, according to one embodiment of the disclosure, conduction of a second operation on the strap tensioning mechanism 245, such as actuation of the control knob 520 of the strap tensioning mechanism 245 that may include rotation in a second angular direction (counter-clockwise (CCW) opposite the first angular rotation for example, causes unwinding of the interconnect 870, which increases the length of the first interconnect segment 872 and allows for de-tensioning, of the first strap 410. Concurrently, rotation of the strap tensioning mechanism 245 in the second angular direction (unwinding of the interconnect 870) increases the length of the second interconnect segment 874 and causes de-tensioning of the second strap 420 concurrently with the first strap 410.

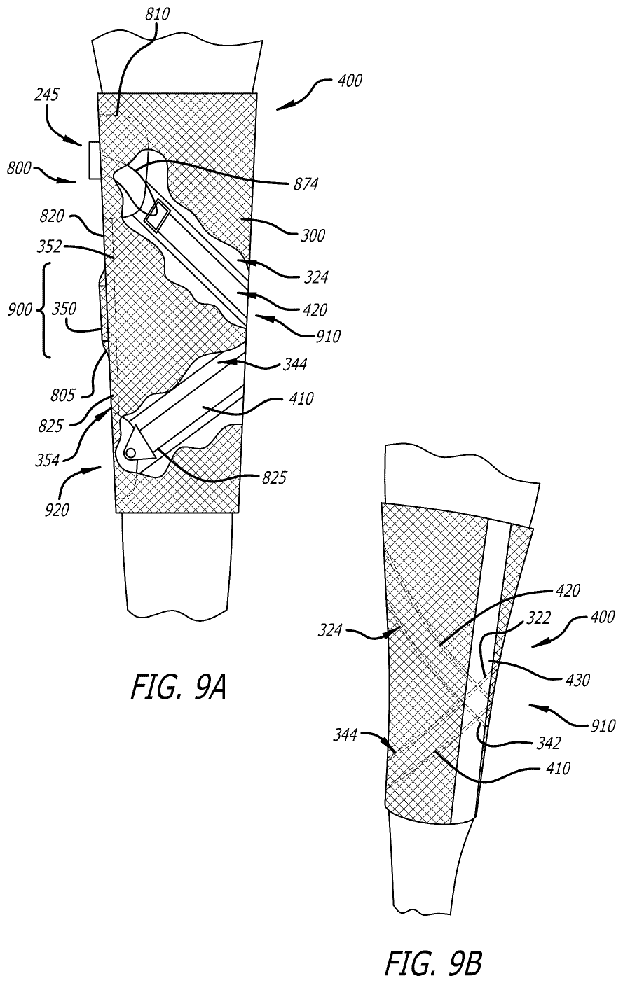

Referring to FIG. 9A, a front perspective view of the sleeve-based orthosis 400 of FIG. 8 with cut-away views of the tunnel segments 324/344, when worn on a right leg by a patient, is shown. Situated above a knee area 900 of the patient, the first end 840 of the second strap 420 is coupled to the first cuff 810 via the second interconnect segment 874 and the strap tensioning mechanism 245. Also, situated below the knee area 900 of the patient, the second end 835 of the first strap 410 is fixedly coupled to the second cuff 815. For this embodiment, when placed on the right leg of the patient, the straps 410 and 420 crisscross along a medial region 910 of the sleeve-based orthosis 400 as shown in FIG. 9B when the hinge 805 and the strut members 820 and 825 are situated along a lateral region 920 of the leg. Alternatively, although not shown, when the sleeve-based orthosis 400 is placed on the left leg of the patient, the straps 410 and 420 would crisscross along the lateral region 920 of the leg as the hinge 805 and the strut members 820 and 825 would be situated along the medial region 910 of the leg.

Referring still to FIG. 9A, the orthotic components 800, such as the hinge 805, the first and second cuffs 810 and 815, and the first and second strut members 820 and 825, along with at least a substantial portion (or the entirety) of the first and second straps 410 and 420, may be encapsulated within the sleeve 300. For example, the hinge 805 may be situated within the third pocket region 350 of the sleeve 300 while first and second strut members 820 and 825 may be encapsulated within tunnel segments 352 and 354. The straps 410 and 420 may be encapsulated in tunnel segments 322, 324, 342, 344 of FIG. 3A, where a portion of the first strap 410 is maintained within the fourth tunnel segment 344 while a portion of the second strap 420 is maintained within the second tunnel segment 324, as shown.

### C. Third Sleeve-Based Orthosis Embodiment

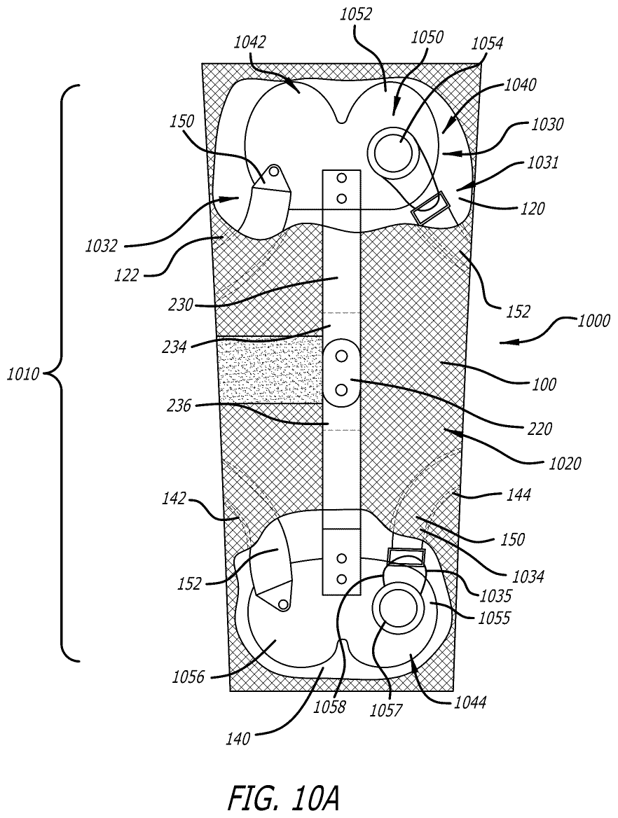

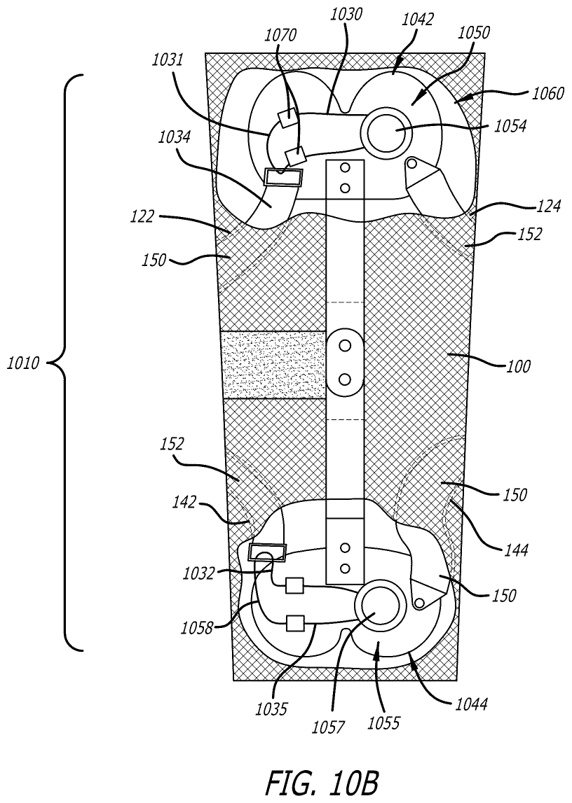

Referring now to FIG. 10A, a third embodiment of a sleeve-based orthosis 1000 with the sleeve 100 folded to expose the cuffs 1042/1044 is shown. The sleeve-based orthosis 1000 includes orthotic components 1010 positioned along a first side (e.g., lateral or medial) of the sleeve-based orthosis 1000, where the orthotic components 1010 include a hinge component 1020 and a plurality of cuffs 1040. Herein, according to this embodiment of the disclosure, the hinge component 1020 is interposed between the first pocket region 120 and the second pocket region 140 of the sleeve 100 of FIG. 1A being utilized by the sleeve-based orthosis 1000. According to one embodiment of the disclosure, the hinge component 1020 may be constructed as the hinge component 215 of FIG. 2A as shown, with hinge 220 and strut members 230 and 232. The strut members 230/232 may be covered by externally welded covers 234/236.

The plurality of cuffs 1040 includes a first cuff 1042 and a second cuff 1044. The first cuff 1042 features a first strap tensioning mechanism 1050 mounted on a cuff plate 1052. The first strap tensioning mechanism 1050 is adapted to adjust the tension of the second strap 152 positioned within the second tunnel segment 124 and the third tunnel segment 142. The second cuff 1044 features a second strap tensioning mechanism 1055 mounted on a second cuff plate 1056. The second strap tensioning mechanism 1055 is adapted to adjust the tension of the first strap 150 positioned within the first tunnel segment 122 and the fourth tunnel segment 144. Hence, the first cuff 1042 is adapted to independently adjust tensioning of the second strap 152 while the second cuff 1044 is adapted to independently adjust tensioning of the first strap 150. This allows different degrees of tensioning can be applied to different straps 150 and 152.

More specifically, a control knob 1054 of the first strap tensioning mechanism 1050 may be actuated (e.g., rotated in a first angular direction such as clockwise CW) to cause winding of a first interconnect 1030, which reduces the length of a first segment 1031 of the interconnect 1030 attached to a first end 1032 of the second strap 152 and causes extension of (tension on) the second strap 152 towards the first cuff 1042. Additionally, the control knob 1054 of the first strap tensioning mechanism 1050 may be actuated (e.g., rotated in a second angular direction such as counter-clockwise CCW, opposite the first angular rotation), which causes unwinding of the interconnect 1030, which increases the length of the first interconnect segment 1031 and allows for de-tensioning of the second strap 152. This effectively reducing the unloading effect on the knee.

Additionally, a control knob 1057 of the second strap tensioning mechanism 1055 may be rotated in a first angular direction (e.g. clockwise CW) causes winding of a second interconnect 1035, which reduces the length of a first segment 1058 of the second interconnect 1035, which is attached to a first end 1034 of the first strap 150 and causes extension of (tension on) the first strap 150 towards the second cuff 1044. Additionally, the control knob 1057 of the second strap tensioning mechanism 1055 may be actuated (e.g., rotated in a second angular direction such as counter-clockwise CCW, opposite the first angular rotation), which causes unwinding of the second interconnect 1035, which increases the length of a segment of the second interconnect 1035 and allows for movement of the first strap 150 away from the second cuff 1044. This reduces tension of the first strap 150 thereby potentially reducing the unloading effect on the knee.

### D. Fourth Sleeve-Based Orthosis Embodiment

Referring now to FIG. 10B, a fourth embodiment of a sleeve-based orthosis 1060 with the folded sleeve representation as in FIG. 10A, except for different configurations of the first cuff 1042 and the second cuff 1044. Herein, the first cuff 1042 features the first strap tensioning mechanism 1050, but it is adapted to adjust the tension of the first strap 150 positioned within the first tunnel segment 122 and the fourth tunnel segment 144. For this implementation, a plurality of guides 1070 are mounted on the first cuff plate 1052. The second cuff 1044 features a second strap tensioning mechanism 1055, which is adapted to adjust the tension of the second strap 152 positioned within the third tunnel segment 142 and anchored at the first cuff 1042. Hence, the first cuff 1042 is adapted to independently adjust tensioning of the first strap 150 while the second cuff 1044 is adapted to independently adjust tensioning of the second strap 152. This allows different degrees of tensioning can be applied to different straps 150 and 152.

With respect to operability, the control knob 1054 of the first strap tensioning mechanism 1050 may be actuated (e.g., rotated in a first angular direction (e.g. clockwise (CW)), which causes winding of the first interconnect 1030 that reduces the length of the first interconnect segment 1031 attached to the first end 1034 of the first strap 150 and applies tension on the first strap 150 towards the first cuff 1042. Additionally, the control knob 1054 of the first strap tensioning mechanism 1050 may be actuated (e.g., rotated in a second angular direction such as a counter-clockwise CCW), which causes unwinding of the interconnect 1030, which allows for movement of the first strap 150 away the first cuff 1042. This effectively reduces tension of the first strap 150 thereby potentially reducing the unloading effect on the knee.

Additionally, the control knob 1057 of the second strap tensioning mechanism 1055 may be actuated, such as rotation in a first angular direction (e.g., clockwise CW), which causes winding of the second interconnect 1035. This reduces the length of the first segment 1058 of the second interconnect 1035, which is attached to the first end 1032 of the second strap 152 and causes tensioning of the second strap 152. Additionally, the control knob 1057 of the second strap tensioning mechanism 1055 may be actuated by rotation in the second (angular direction (e.g., counter-clockwise CCW), which causes unwinding of a segment of the second interconnect 1035, which lengths the second interconnect 1035 and allows for movement of the second strap 152 away the second cuff 1044. This reduces tension of the second strap 152 thereby potentially reducing the unloading effect on the knee.

### E. Perspective Views of Dual-Controlled Sleeve-Based Orthoses

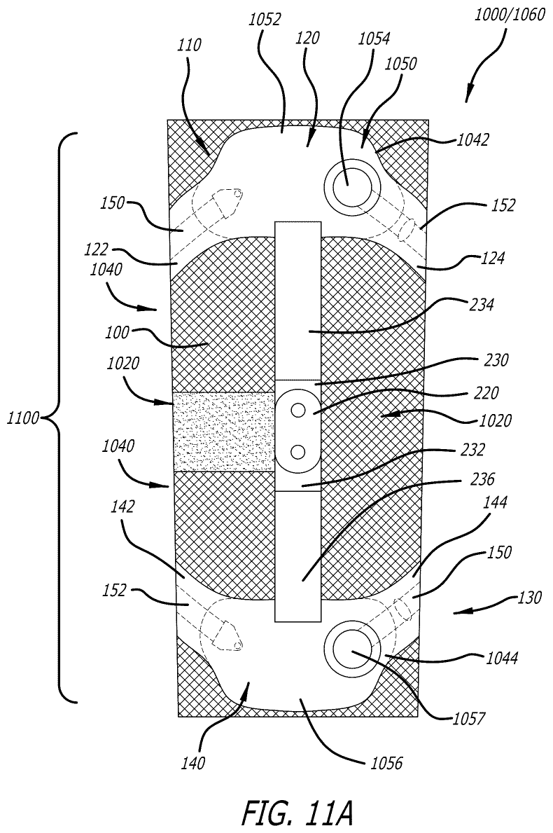

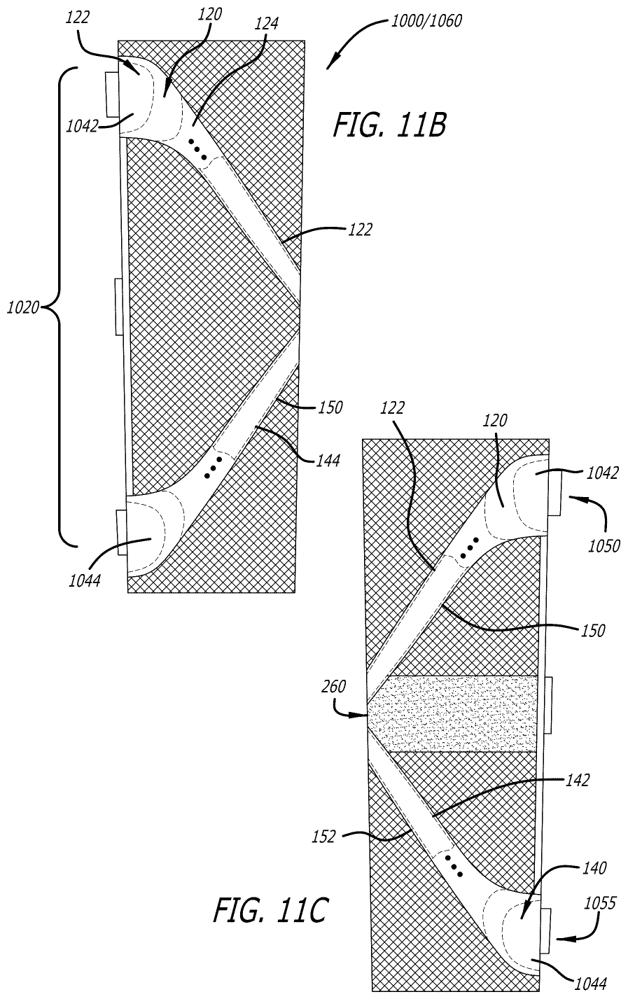

Referring now to FIG. 11A, an exemplary side perspective of the sleeve-based orthosis 1000/1060 with cut-away views of the pocket regions 120/140 and placed into an operational state is shown. Herein, sleeve-based orthosis 1000/1060 features the sleeve 100 in which its edges are folded towards to other to produce a substantially cylindrical form. As a result, the first pocket region 120 of the first pathway 110 is intended to be positioned above the knee and the second pocket region 140 is intended to be positioned below the knee (e.g., resting over the tibia).

The sleeve-based orthosis 1000/1060 features a dynamic unloading system 1100, which includes the hinge component 1020 and the plurality of cuffs 1040, which includes the first cuff 1042 and the second cuff 1044. Herein, the hinge component 1020 is interposed between the first pocket region 120 and the second pocket region 140 of the sleeve 100 and exposed. According to one embodiment of the disclosure, the hinge component 1020 includes (i) the hinge 220 configured to allow for controlled movement of a joint to mimic its natural motion to control extension and/or flexion, and/or (ii) the pair of strut members 230/232 coupled to the hinge 220. The strut members 230/232 may include a corresponding welded cover 234/236 positioned over each of the strut members 230/232.

As further shown in FIG. 11A, the first cuff 1042 features the cuff plate 1052 and the strap tensioning mechanism 1050 mounted thereon. The cuff plate 1052 is positioned to reside within the first pocket region 120, and the control knob 1054 extends through a cut-out within an exterior surface of the sleeve 100. Where the sleeve-based orthosis 1000 of FIG. 10A is utilized, as shown, the control knob 1054 is adapted to independently adjust the tension of the second strap 152 positioned within the second tunnel segment 124 and the third tunnel segment 142. An end of the first strap 150 is fixedly attached to the cuff plate 1052. Alternatively, where the sleeve-based orthosis 1060 of FIG. 10B is utilized (not shown), the control knob 1057 would be adapted to independently adjust the tension of the first strap 150 positioned within the first tunnel segment 122 and the fourth tunnel segment 144. An end of the second strap 152 would be fixedly attached to the cuff plate 1056.

Additionally, as shown in FIG. 11A, the second cuff 1044 features the second cuff plate 1056 and the second strap tensioning mechanism 1055 mounted thereon. The second cuff plate 1056 is positioned to reside within the second pocket region 140, and the control knob 1057 extends through a cut-out within an exterior surface of the sleeve 100. Where the sleeve-based orthosis 1000 of FIG. 10A is utilized, as shown, the control knob 1057 is adapted to independently adjust the tension of the first strap 150 positioned within the fourth tunnel segment 144 and the first tunnel segment 122. However, although not shown, where the sleeve-based orthosis 1060 of FIG. 10B is utilized, the control knob 1054 would be adapted to independently adjust the tension of the second strap 152 positioned within the third tunnel segment 142 and the second tunnel segment 124.

Referring to FIG. 11B, a front perspective view of the sleeve-based orthosis 1000/1060 is shown. For this embodiment, the hinge component 1020 may be positioned on a lateral side when placed on a right leg of the patient. But, given its pseudo-universal design, the hinge component 1020 may be positioned on a medial side when the sleeve-based orthosis 1000/1060 is placed on a left leg of the patient, where this perspective view would be rear perspective view in lieu of the front perspective view.

As shown, for this embodiment, the second tunnel segment 124 extends diagonally downward from the first pocket region 120 toward a mid-medial region of the sleeve-based orthosis 1000/1060. Also, the fourth tunnel segment 144 extends diagonally upward from the second pocket region 140 toward the mid-medial region of the sleeve-based orthosis 1000/1060. Complementary in orientation, as shown in FIG. 11C, the first tunnel segment 122 extends diagonally downward from the first pocket region 120 toward the mid-medial region 250 on a side of the sleeve-based orthosis 1000/1060 opposite the hinge component 1020. Also, the third tunnel segment 142 extends diagonally upward from the second pocket region 140 toward the mid-medial region 250 of the sleeve-based orthosis 1000/1060.

Hence, the connective framework of the sleeve-based orthosis 1000/1060 is dependent on the type of cuff deployed. For instance, for the sleeve-based orthosis 1000, the second strap 152 is (i) adjustably coupled to the first strap tensioning mechanism 1050 situated on the first cuff 1042 maintained in the first pocket region 120, (ii) fixedly coupled to the second cuff 1044 maintained within the second pocket region 140, and (ii) substantially encapsulated within the second tunnel segment 124 and the third tunnel segment 142. Additionally, the first strap 150 is (i) adjustably coupled to the second strap tensioning mechanism 1055 situated on the second cuff 1044 maintained in the second pocket region 140, (ii) fixedly coupled to the first cuff 1042 maintained within the first pocket region 120, and (ii) substantially encapsulated within the fourth tunnel segment 144 and the first tunnel segment 122.

Similarly, for the sleeve-based orthosis 1060, the second strap 152 is (i) adjustably coupled to the first strap tensioning mechanism 1050 situated on the first cuff 1042 maintained in the first pocket region 120, (ii) fixedly coupled to the second cuff 1044 maintained within the second pocket region 140, and (iii) substantially encapsulated within the tunnel segment 124 and the tunnel segment 142. Additionally, the first strap 150 is (i) adjustably coupled to the second strap tensioning mechanism 1055 situated on the second cuff 1044 maintained in the second pocket region 140, (ii) fixedly coupled to the first cuff 1042 maintained within the first pocket region 120, and (ii) substantially encapsulated within the tunnel segment 122 and the tunnel segment 144.

As further shown FIG. 11C, the openings 160/162 formed on the pathways 110 and 130 of the sleeve 100 of FIG. 1A would be aligned to provide an intersection point between the first strap 150 and the second strap 152, where the adjustable straps 150/152 intersect at the mid-medial region 250. Although not shown, fasteners may be attached to the exterior surface of the sleeve to retain in its substantially cylindrical form. Additionally, or as the alternative feature, the band of thermoplastic material 260 may be positioned longitudinally along the second side (e.g., medial side) to provide additional stability and support after the thermoplastic material is heated above its thermal melting threshold and subsequent cooling to transform into a semi-rigid form.

### IV. General Architecture-Cuffs

### A. First Cuff Embodiment

Referring now to FIG. 12, a first embodiment of the first cuff 242 deployed within the sleeve-based orthoses 200/400 of FIGS. 5 & 8 is shown. The first cuff 242 includes a cuff plate 1200 with the strap tensioning mechanism 245, which is mounted on a top surface 1210 of the cuff plate 1200. The strap tensioning mechanism 245 features the control knob 520 which, when manipulated, is used to adjust the degree of extension (or retraction) of the interconnect 530 attached to the first and second straps 1220/1230. Depending on the sleeve-based orthosis 200 or 400, the first and second straps 1220/1230 may be representative of straps 150/152 of FIG. 5 or straps 410/420 of FIG. 8.

As shown in FIG. 12, for the sleeve-based orthosis 200, the interconnect 530 is arranged in a loop configuration, where the length of the interconnect 530 is adjusted based on rotation of the control knob 520. For example, illustrating the first cuff 242 is configured to be deployed in the sleeve-based orthosis 200 of FIG. 5, the first end 540 of the first strap 150 is coupled to the first attachment member 545 (e.g., D-ring) and the interconnect 530 is coupled to the first attachment member 545. The interconnect 530 is maintained in the loop configuration by multiple guide members 1240, where a first set of guide members 1241 and 1242 is attached to the cuff plate 1200 to retain positioning of a first lead section 1250 and a first return section 1252 of the interconnect 530 coupled to the first attachment member 545. The multiple guide members 1240 further include a second set of guide members 1243 and 1244, which is attached to the cuff plate 1200 to retain positioning of a second lead section 1254 and a second return section 1256 of the interconnect 530 coupled to a second attachment member 546.

Based on this configuration, rotation of the control knob 520 in a first direction (CW) causes concurrent tensioning of both the first strap 150 and the second strap 152. Conversely, rotation of the control knob 520 in a second direction (CCW) or pulling the knob to fully release interconnect causes concurrent de-tensioning of both the first strap 150 and the second strap 152.

### B. Second Cuff Embodiment

Referring to FIGS. 13A-13B, a second embodiment of a first cuff 1300 deployed within the sleeve-based orthoses 200/400 of FIGS. 5 & 8 is shown. As an illustrative example, for the sleeve-based orthosis 400, the first cuff 1300 includes a cuff plate 1305 with dual mounting engagements 1310 and 1320 integrated on a front or top surface 1330 and on opposite sides of the cuff plate 1305. Each mounting engagement 1310 and 1320 is configured for coupling of the strap tensioning mechanism thereto. Herein, the first mounting engagement 1310 is situated to allow for placement of the strap tensioning mechanism in front of a coronal plane toward an anterior area of the patient when the first cuff 1300 is mounted on a right leg of the patient, or as an alternative embodiment, behind a coronal plane toward a posterior area of the patient when the first cuff 1300 is mounted on the right leg of the patient. Similarly, the second mounting engagement 1320 is situated to allow for placement of the strap tensioning mechanism in front of a coronal plane toward an anterior area of the patient when the first cuff 1300 is mounted on a left leg of the patient, or as an alternative embodiment, behind a coronal plane toward a posterior area of the patient when the first cuff 1300 is mounted on the left leg of the patient. As a result, a single cuff design may support multiple deployments.

As further shown in FIGS. 13A-13B, the first cuff 1300 further includes a plurality of guide rails, including of at least a first set (one or more) of guide rails 1340 and a second set of guide rails 1345, which are configured to secure and maintain a prescribed orientation of a first lead section 1350 and a second lead section 1354 of the interconnect 870. The first set of guide rails 1340 is configured to secure and maintain a prescribed orientation of the first lead section 1350 that is coupled to the first end 830 of the first strap 410. The second set of guide rails 1345 is configured to secure and maintain a prescribed orientation of the second lead section 1354 of the interconnect 870. As shown, the sets of guide rails 1340 and 1345 are integrated (molded) as part of the first cuff 1300.

Additionally, the first cuff 1300 may feature a third set of guide rails 1360, which operate to assist the first set of guide rails 1340 and the second set of guide rails 1345 to retain retaining positioning of the leads section 1350/1354 and return sections 1352/1356 of the interconnect 870 as shown in FIG. 13B. Although these sets of guide rails 1340/1345/1360 are performed into the cuff plate 1305 as shown, according to another embodiment of the disclosure, the sets of guide rails 1340/1345/1360 may be separate components that are affixed to the top surface 1330 of the first cuff 1300.

As further shown in FIG. 13A, the first cuff 1300 may be configured with a prescribed region 1370 defined by the raised border 1375 formed along the top surface 1330 of the cuff plate 1305 to receive an edge of the first strut member 230.

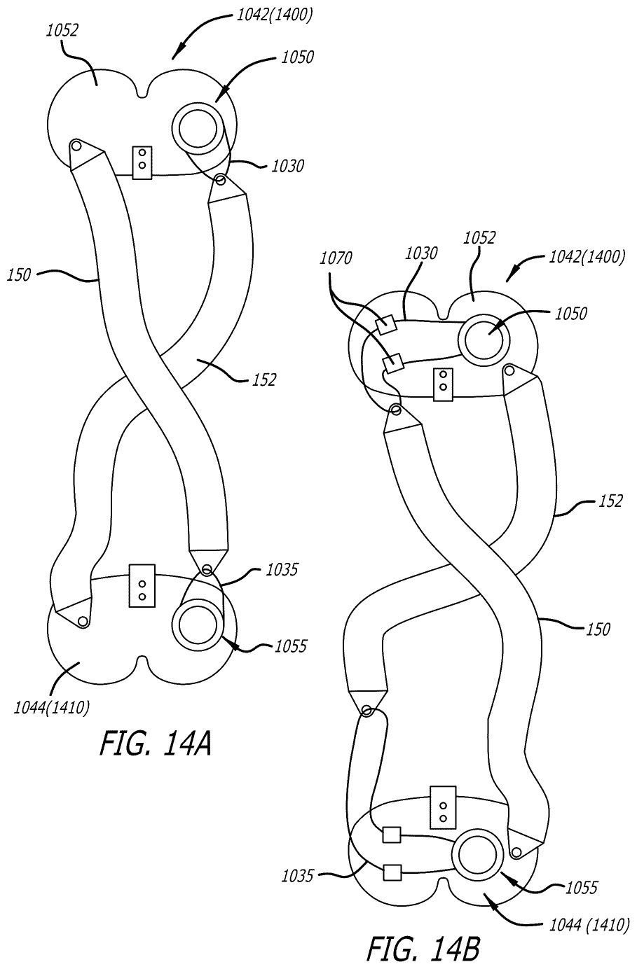

Referring to FIG. 14A, a third embodiment of the cuff implementations of FIGS. 10A-10B is shown, each having a strap tensioning mechanism with different interconnect connectivity to support different strap controllability. Herein, a first cuff 1400, such as the first cuff 1042 of FIG. 10A for example, features the strap tensioning mechanism 1050 mounted on a cuff plate. The strap tensioning mechanism 1050 is adapted to adjust the tension of an anteriorly positioned strap (e.g., second strap 152) via interconnect 1030 independent of an adjustment of the other strap (e.g., first strap 150), which is managed by a second cuff 1410 such as the second cuff 1044 as described in FIG. 10A. A mirror configuration is conducted on the second cuff 1410 to adjust tension of the posteriorly positioned strap (e.g., first strap 150) via the interconnect 1035.

Alternatively, as further shown in FIG. 14B, the first cuff 1400 may be configured with the strap tensioning mechanism 1050, but it is adapted to adjust the tension of a posteriorly positioned strap (e.g., first strap 150) in which the plurality of guides 1070 are mounted on the cuff plate 1052 to retain positioning of the interconnect 1030 as shown in FIG. 10B. A mirror configuration is conducted on the second cuff 1410 to adjust tension of the anteriorly positioned strap (e.g., second strap 152) via the interconnect 1035.

### V. General Architecture-Hinge

Referring now to FIG. 15, an illustrative embodiment of mechanical components of the hinge component 215 of FIG. 2A is shown, which include the hinge 220 and the pair of strut members 230 and 232. For this embodiment, the hinge 220 includes the first and second fastening elements 1511 and 1512; the first and second hinge covers 1513 and 1514; a hinge front plate 1500; a hinge back plate 1510; a first set of washers 1520 positioned between the hinge front plate 1500 and the strut members 230 and 232; a second set of washers 1530 positioned between the hinge back plate 1510 and the strut members 230 and 232; an optional extension stop component 1540; and/or an optional flexion stop component 1550. A coupling path 1560 illustrates an attachment sequence for the first fastening element 1511 to form the first axis of rotation 1521 while a second coupling path 1570 illustrates an attachment sequence for the second fastening element 1512 to form the second axis of rotation 1522.

More specifically, the first hinge cover 1513 includes a first base component 1580 and a first arm component 1585 while the second hinge cover 1514 includes a second base component 1590 and a second arm component 1595. Herein, according to this embodiment of the disclosure, the first hinge cover 1513 is rotationally coupled to the hinge front plate 1500 by insertion of the first fastening element 1511 into a recessed aperture 1581 formed within the first base component 1580. An end section 1582 of the first base component 1580 features a recessed area 1584 sized to receive a detent (protrusion) 1593 situated within the second arm component 1595 of the second hinge cover 1514. The first arm component 1585 of the first hinge cover 1513 includes a first detent (protrusion) 1583, which is sized for retention within a recessed area 1594 of the second hinge cover 1514.

Similarly, the second hinge cover 1514 is rotationally coupled to the hinge front plate 1500 by insertion of the second fastening element 1512 into a recessed aperture 1591 formed within the second base component 1590. An end section 1592 of the second base component 1590 features the recessed area 1594 sized to receive the first detent 1583 situated within the first arm component 1585 of the first hinge cover 1513. The second arm component 1595 of the second hinge cover 1514 includes the detent (protrusion) 1593, which is sized for retention within the recessed area 1584 of the first hinge cover 1513.

Referring still to FIG. 15, the hinge front plate 1500 is configured with a first set of stability notches (e.g., first stability notch 1502) may be formed along a first edge 1503 of the hinge front plate 1500. The first stability notch 1502 is formed to engage with a first stability element 1542 positioned on a side surface 1544 of the extension stop component 1540. This engagement is designed to prevent (or at least substantially mitigate) vertical movement of the extension stop component 1540 during usage of the sleeve-based orthosis 200/400/1000/1060. Additionally, a second set of stability notches (e.g., second and third stability notches 1505) may be formed along a second edge 1506 of the hinge front plate 1500. These stability notches 1505 are formed to engage with a corresponding set of stability elements 1552 positioned on a side surface 1554 of the flexion stop component 1550. As shown, the number of stability elements 1542 positioned on the extension stop component 1540 may differ from the number of stability elements 1552 positioned on the flexion stop component 1550 for easy identification as to whether the stop component is designed to control extension or flexion.

The extension stop component 1540 is positioned to reside within a first (anterior) spacing 1546 formed between first (front-facing) edges of the strut members 230 and 232, with the first stability element 1542 being positioned to engage with the first stability notch 1502 when the extension stop component 1540 is placed within the first spacing 1546. Similar in construction, the flexion stop component 1550 is formed to reside within a second (posterior) spacing 1556 formed between second (back-facing) edges of the strut members 230 and 232, with the set of stability elements 1552 being positioned to engage with the stability notches 1505 when the flexion stop component 1550 is placed within the second spacing 1556.

During extension, clockwise (forward) rotational movement of the first strut member 230 along with clockwise (backward) rotational movement of the second strut member 232, the first (front-facing) edges of the strut members 230/232 engage the extension stop component 1540, which is sized to allow for a prescribed degree of extension. During flexion, counter-clockwise (backward) rotational movement of the first strut member 230 along with counter-clockwise (forward) rotational movement of the second strut member 232, the second (back-facing) edges of the strut members 230/232 engage the flexion stop component 1550, which is sized with a length to allow for a prescribed degree of flexion.

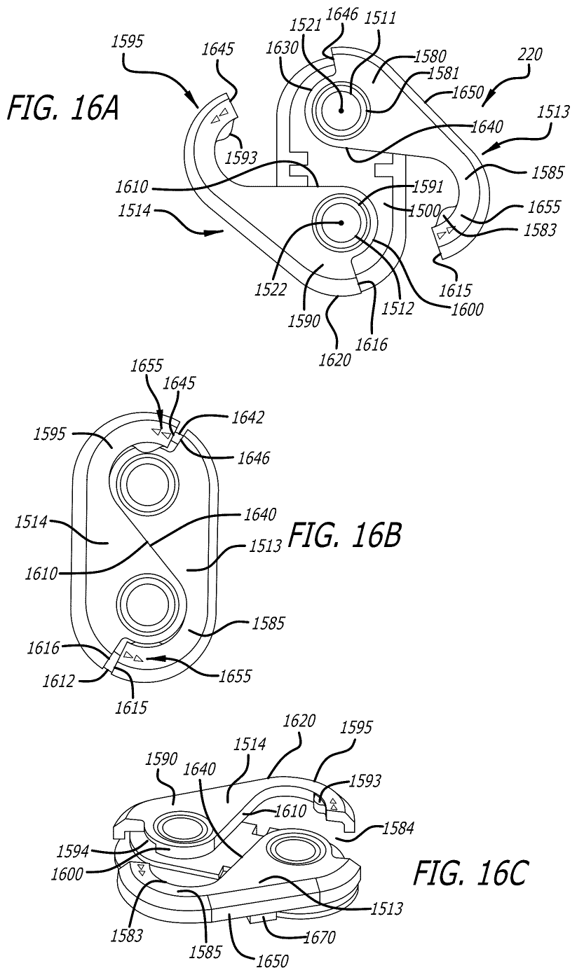

Referring now FIG. 16A, an overhead planar view of a first exemplary embodiment of the hinge 220 of FIG. 15 placed into a partially open (or unlocked) state is shown. Herein, the hinge 220 features the first hinge cover 1513 and the second hinge cover 1514. The first hinge cover 1513 is mounted on the hinge front plate 1500 (see FIG. 15) and rotational about the first axis of rotation 1521. The first axis of rotation 1521 is colinear with the first fastening element 1511. Similarly, the second hinge cover 1514 is mounted on the hinge front plate 1500 and rotational about the second axis of rotation 1522. The second axis of rotation 1522 is colinear with the second fastening element 1512.

As shown, the first hinge cover 1513 includes the first base component 1580 and the first arm component 1585 while the second hinge cover 1514 includes the second base component 1590 and the second arm component 1595. Herein, the first base component 1580 corresponds to a section of the first hinge cover 1513 that surrounds the aperture 1581 adapted to receive the first fastening element 1511. The first arm component 1585, corresponding to a distal portion of the first hinge cover 1513, is configured with a concave-shaped curvature to (i) partially align with an angular (convex) segment 1600 of an inner edge 1610 of the second hinge cover 1514 and (ii) provide a gap 1612 (see FIG. 16B) between a distal end 1615 for the first arm component 1585 of the first hinge cover 1513 and a proximal end 1616 for the second base component 1590 of the second hinge cover 1514 that connects the inner edge 1610 with the outer edge 1620.

Similarly in construction, the second base component 1590 corresponds to a section of the second hinge cover 1514 that surrounds the aperture 1591 adapted to receive the second fastening element 1512. The second arm component 1595 corresponds to a section of the second hinge cover 1514 having a concave-shaped curvature that (i) partially aligns with an angular segment 1630 of an inner edge 1640 of the first hinge cover 1513 and (ii) provides a gap 1642 (see FIG. 16B) between a distal end 1645 of the second hinge cover 1514 and a proximal end 1646 of the first hinge cover 1513 that connects the inner edge 1640 with an outer edge 1650.

As a result, as shown in FIG. 16B, when the hinge 220 is placed into a closed (locked) state, the inner edge 1640 of the first hinge cover 1513 is complementary to and aligned with the inner edge 1610 of the second hinge cover 1514. Also, the first gap 1612 is formed between the proximal end 1616 of the second hinge cover 1514 and the distal end 1615 of the first hinge cover 1513 featuring indicia 1655 identifying a direction of movement needed from the first gap 1612 to unlock the hinge 220. The second gap 1642 is formed between the proximal end 1646 of the first hinge cover 1513 and the distal end 1645 of the second hinge cover 1514 featuring indicia 1656 identifying a direction of movement needed from the second gap 1642 to unlock the hinge 220.

As shown in FIGS. 16A-16B, the inner edge 1640 of the first hinge cover 1513 includes the detent (protrusion) 1583 positioned toward at the distal end 1615 of the first arm component 1585 while the inner edge 1610 of the second hinge cover 1514 includes a detent (protrusion) 1593 positioned toward at the distal end 1645 of the second arm component 1595. Given that the first hinge cover 1513 and the second hinge cover 1514 may have mirror constructions, where one hinge cover (e.g., hinge cover 1513) is inclined inwardly and the other hinge cover (e.g., hinge cover 1514) is inclined outwardly. However, the below discussion of the formulation of the detent 1583 for the first hinge cover 1513 is applicable to the formulation of the detent 1593 for the second hinge cover 1514.

More specifically, the detent 1583 is configured to facilitate attachment of the first hinge cover 1513 to the second hinge cover 1514 while the detent 1593 is configured to facilitate attachment of the second hinge cover 1514 to the first hinge cover 1513. More specifically, the detent 1583 is sized to reside within the recessed area 1594 formed within the angular segment 1600 of the inner edge 1610 of the second hinge cover 1514 when the hinge 220 is placed in a closed (locked) state as shown in FIGS. 16B-16C. As the hinge 220 is placed into a closed (locked) state, prior to resting with the recessed area 1594, the detent 1583 comes into contact with an angular segment 1600 of the inner edge 1610 forming part of the second base component 1590, which causes a slight increase in the curvature of the first arm component 1585 (e.g., raised or elongation of the concave-shaped first arm component 1585) until the detent 1583 clears an apex formed by the angular segment 1600 and returns back to its “at rest” curvature (length) to reside within the recessed area 1594. The same operations occur for the detent 1593 placed toward the distal end 1645 of the second hinge cover 1514.

Referring now to FIG. 16C, the outer edge 1650 of the first hinge cover 1513 includes a flange 1670 positioned to overlap a portion of an entry area for the stop component 1540 of FIG. 15 when the first hinge cover 1513 is placed into a closed (locked) state. This overlapping flange 1670 is adapted to preclude removal of the stop component 1540, unless the hinge 220 is placed into an open (or unlocked) state in which both the first and second hinge covers 1513 and 1514 are rotated in a first direction (e.g., counterclockwise “CCW” direction) concurrently. Similarly, the outer edge 1620 of the second hinge cover 1514 features a flange (not shown) positioned to overlap a portion of an entry area for another stop component 1550 of FIG. 15. This overlapping flange is adapted to preclude removal of the stop component 1550 unless the hinge 220 is placed into an open (or unlocked) state in which both the first and second hinge covers 1513 and 1514 are rotated in the first direction (e.g., CCW) concurrently.

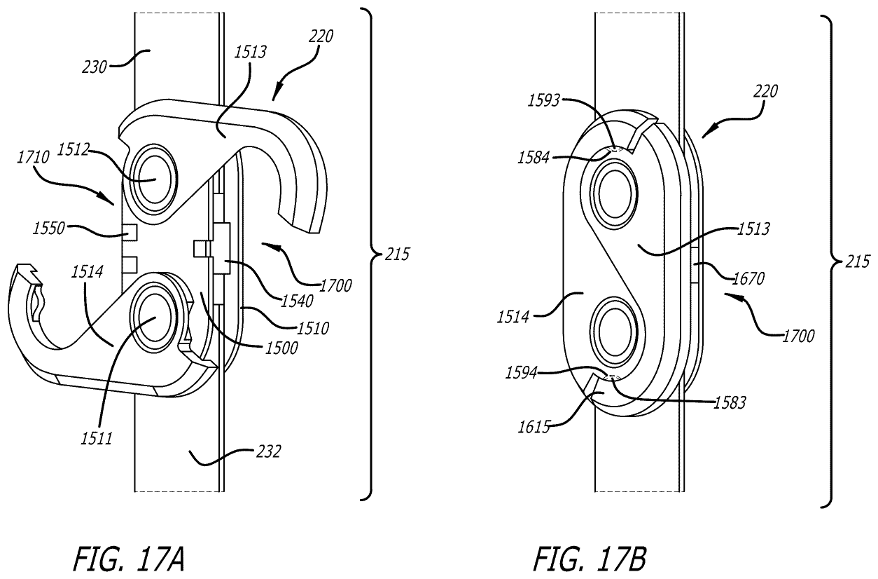

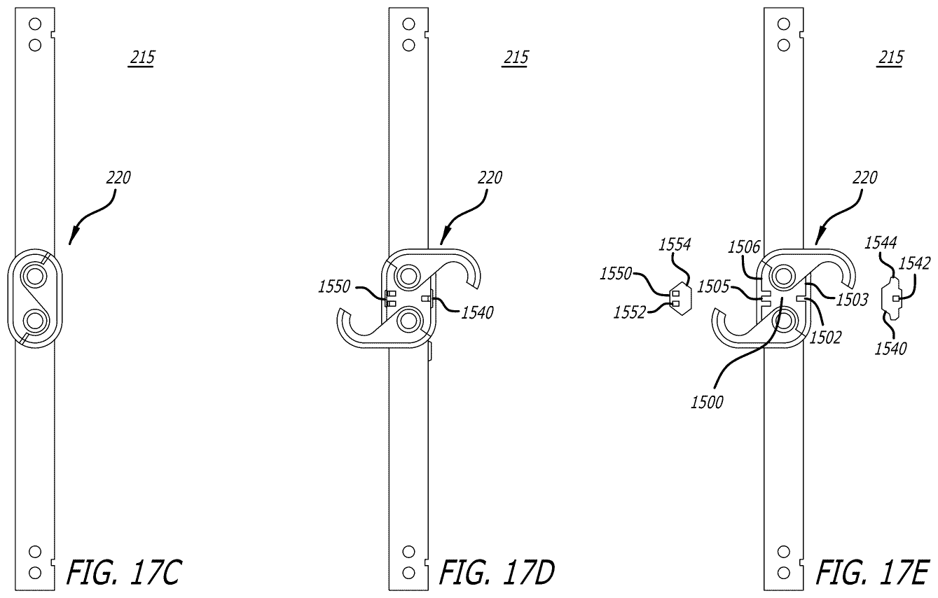

Referring now to FIG. 17A, a perspective view of an illustrative embodiment of the hinge 220 of FIG. 15 oriented in a resting (non-flexed) state and the hinge 220 placed in an open (or unlocked) state. To retain the hinge 220 to the strut members 230/232, the fastening elements 1511 and 1512 are placed through apertures formed in the hinge covers 1513 and 1514, the hinge front plate 1500, strut members 230 and 232, and the hinge back plate 1510. Entry areas 1700 and 1710 are formed between ends of the strut members 230 and 232 to receive the stop component 1540 and/or the stop component 1550. The hinge covers 1513 and 1514 are rotated to expose the entry areas 1700 and 1710 for insertion of the components 1540 and 1550 when desired.

Referring to FIG. 17B, a perspective view of an illustrative embodiment of the hinge 220 of FIG. 15 oriented in a resting (non-flexed) state and the hinge 220 placed in a closed (locked) state. Herein, the hinge covers 1513 and 1514 are rotated until the detent 1583 positioned towards the distal end of the first hinge cover 1513 is positioned to rest within the recessed area 1594 formed within the second base component 1590 and the detent 1593 positioned towards the distal end of the second hinge cover 1514 is positioned to rest within the recessed area 1584 similar to recessed area 1594 formed within the first base component 1580.

Referring to FIGS. 17C-17E, perspective series of views of illustrative embodiments of the hinge 220 of FIGS. 17A-17B are shown, where the hinge 220 is oriented in a resting (non-flexed) state with both the stop (extension) component 1540 and the stop (flexion) component 1550 being removed from the hinge 220 placed in an open (or unlocked) state. The hinge 220 transitions from a closed (or locked) state (see FIG. 17C) to an open (or unlocked) state (see FIG. 17D). As shown in FIG. 17E, the hinge front plate 1500 is configured with the first set of stability notches (e.g., first stability notch 1502) formed along the first edge 1503 and the second set of stability notches (e.g., second and third stability notches 1505) may be formed along the second edge 1506 of the hinge front plate 1500. Upon removal of the stop component 1540, the first stability element 1542 positioned on the side surface 1544 of the stop component 1540 is disengaged from the first stability notch 1502. Upon removal of the stop component 1550, the stability elements 1552 positioned on the side surface 1554 of the stop component 1550 are disengaged from the second and third stability notches 1505. As shown, the number of stability elements 1542 positioned on the stop component 1540 may differ from the number of stability elements 1552 positioned on the stop component 1550 for easy identification as to the type of stop component (e.g., extension or flexion), although different differentiation techniques (e.g., color, markings, etc.) may be used as well.

In the foregoing description, the invention is described with reference to specific exemplary embodiments thereof. Hence, it will be evident that certain components may be deployed within different types of sleeve-based orthoses and various modifications and changes may be made thereto without departing from the broader spirit and scope of the invention as set forth in the appended claims.

What is claimed is:

1. An orthopedic sleeve comprising:
a sleeve body; and
one or more tunnel segments formed within the sleeve body, wherein each of the one or more tunnel segments is configured to encapsulate a portion of a strap that is adapted to apply an unloading effect on a body part stabilized by the orthopedic sleeve.

2. The orthopedic sleeve of claim 1, wherein the one or more tunnel segments are positioned to concentrate at a desired side area of the body part being a knee to unload forces against the knee.

3. The orthopedic sleeve of claim 1 further comprising:
a plurality of pockets formed on the sleeve body, the plurality of pockets include a first pocket and a second pocket, wherein the one or more tunnel segments are formed in the sleeve body between the first pocket and the second pocket.

4. The orthopedic sleeve of claim 3, wherein the one or more tunnel segments includes a first tunnel segment extending downwardly in a first direction from the first pocket, a second tunnel segment extending downwardly in a second direction, being different from the first direction, from the first pocket, a third tunnel segment extending upwardly to become aligned with the first tunnel segment, and a fourth tunnel segment extending upwardly to become aligned with the second tunnel segment.

5. The orthopedic sleeve of claim 4, wherein the first tunnel segment, the second tunnel segment, the third tunnel segment, and the fourth tunnel segment are positioned to intersect at a medial or lateral side of the orthopedic sleeve.

6. The orthopedic sleeve of claim 5, wherein the first tunnel segment is adapted to encapsulate a first portion of a first strap, the second tunnel segment is adapted to encapsulate a first portion of a second strap, the third tunnel segment is adapted to encapsulate a second portion of the second strap, and the fourth tunnel segment is adapted to encapsulate a second portion of the first strap.

7. The orthopedic sleeve of claim 6, wherein the first strap and the second strap crisscross at the medial or lateral side of the orthopedic sleeve.

8. The orthopedic sleeve of claim 1, wherein the one or more tunnel segments are knitted to be formed within the sleeve body or the one or more tunnel segments are formed by a layer of material, separate from the sleeve body, attached to a surface of the sleeve body to form the one or more tunnel segments.

9. The orthopedic sleeve of claim 1 further comprising a band of thermoplastic material positioned longitudinally along the sleeve body to provide additional support after the thermoplastic material is heated above its thermal melting threshold and subsequent cooled to assume a semi-rigid form on the orthopedic sleeve being of a flexible form.

10. An orthopedic sleeve operating for positioning to surround a knee of a patient, the orthopedic sleeve comprising:
a sleeve body; and
a plurality of tunnel segments formed within the sleeve body, wherein each of the plurality of tunnel segments is configured to encapsulate a portion of a strap to which a prescribed amount of tension can be initiated to apply an unloading effect on the knee stabilized by the orthopedic sleeve.

11. The orthopedic sleeve of claim 10, wherein the plurality of tunnel segments are positioned to concentrate at a desired side area of the knee the unloading effect being lateral or medial forces against the knee.

12. The orthopedic sleeve of claim 10 further comprising:
a plurality of pockets formed on the sleeve body, the plurality of pockets include a first pocket and a second pocket, wherein the plurality of tunnel segments are formed in the sleeve body between the first pocket and the second pocket.

13. The orthopedic sleeve of claim 12, wherein the plurality of tunnel segments includes a first tunnel segment extending downwardly in a first direction from the first pocket, a second tunnel segment extending downwardly in a second direction, being different from the first direction, from the first pocket, a third tunnel segment extending upwardly to become aligned with the first tunnel segment, and a fourth tunnel segment extending upwardly to become aligned with the second tunnel segment.

14. The orthopedic sleeve of claim 13, wherein the first tunnel segment, the second tunnel segment, the third tunnel segment, and the fourth tunnel segment are positioned to intersect at a medial side or a lateral side of a knee covered by the orthopedic sleeve.

15. The orthopedic sleeve of claim 14, wherein the first tunnel segment is adapted to encapsulate a first portion of a first strap, the second tunnel segment is adapted to encapsulate a first portion of a second strap, the third tunnel segment is adapted to encapsulate a second portion of the second strap, and the fourth tunnel segment is adapted to encapsulate a second portion of the first strap.

16. The orthopedic sleeve of claim 15, wherein the first strap and the second strap crisscross at the medial side or at the lateral side of the orthopedic sleeve.

17. The orthopedic sleeve of claim 10, wherein the plurality of tunnel segments are knitted to be formed within the sleeve body or the plurality of tunnel segments are formed by a layer of material, separate from the sleeve body, sewn to a surface of the sleeve body to form the plurality of tunnel segments.

18. The orthopedic sleeve of claim 10 further comprising a band of thermoplastic material positioned longitudinally along the sleeve body to provide additional support after the thermoplastic material is heated above its thermal melting threshold and subsequent cooled to assume a semi-rigid form on the orthopedic sleeve being of a flexible form.
